(19) United States
(12) Patent Application Publication
(10) Pub. No.: US 2005/0272784 A1
(43) Pub. Date: Dec. 8, 2005

(54) Inhibitors of bacterial Type III protein secretion systems

(76) Inventors: Xiaobing Li, Flemington, NJ (US); William V. Murray, Belle Mead, NJ (US); Mark J. Macielag, Branchburg, NJ (US); Qunying Guan, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/123,977

(22) Filed: May 6, 2005

Related U.S. Application Data
(60) Provisional application No. 60/568,851, filed on May 7, 2004.

(51) Int. Cl. A61K 31/4245; C07D 413/02

(52) U.S. Cl. 514/364; 514/374; 548/131

(57) ABSTRACT

In accordance with the present invention, compounds that inhibit Type III protein secretion have been identified, and methods for their use provided. In one aspect of the invention, compounds useful in the inhibition of Type III protein secretion and/or in the treatment and prevention of bacterial infections, particularly Gram-negative bacterial infections, are provided. In another aspect of the invention, methods are provided for the inhibition of Type III protein secretion and/or the treatment and prevention of bacterial infections, particularly Gram-negative bacterial infections using the compounds of the invention.

### CROSS REFERENCE TO RELATED APPLICATIONS

This applications claims the benefit under 35 U.S.C. 119(e) of provisional application, Ser. No. 60/568,851, filed May 7, 2004.

### FIELD OF THE INVENTION

The subject invention relates to novel anti-microbial compounds, their compositions and their uses.

### BACKGROUND OF THE INVENTION

Type III protein secretion systems are an essential virulence determinant of most pathogenic Gram-negative bacteria, including Salmonella, Shigella, Yersinia, Pseudomonas aeruginosa, and enteropathogenic Escherichia coli. The Type III virulence mechanism consists of a secretion apparatus, consisting of about 25 proteins, and a set of effector proteins released by this apparatus. Following activation by intimate contact with a eukaryotic cell membrane, the effector proteins are injected into the host cell, where they subvert the signal transduction machinery and lead to a variety of host cell responses. This virulence mechanism plays a key role in establishing and maintaining an infection and in the resulting pathophysiological sequelae, such as diarrhea, chronic lung inflammation, and septicemia.

Certain protein components of the Type III secretion apparatus are highly conserved among bacterial pathogens, and as such represent suitable targets for therapeutic intervention. Inhibitors of Type III protein secretion are expected to be useful as prophylactic agents (i.e., to prevent the onset of infection by Gram-negative bacteria) or as drugs to treat an existing bacterial infection, either with or without an anti-bacterial agent.

There remains a need to develop, characterize, and optimize lead molecules for the development of novel anti-bacterial drugs. Accordingly, it is an object of the present invention to provide such compounds.

### SUMMARY OF THE INVENTION

In accordance with the present invention, compounds that inhibit Type III protein secretion have been identified, and methods for their use provided.

In one aspect of the invention, compounds of Formula (I) are provided which are useful in the inhibition of Type III protein secretion and/or in the treatment and prevention of bacterial infection, particularly Gram-negative bacterial infection.

In another aspect of the invention, methods are provided for the inhibition of Type III protein secretion and/or in the treatment and prevention of bacterial infection, particularly Gram-negative bacterial infection using the compounds described herein.

In one embodiment, the invention is directed to methods for inhibiting Type III protein secretion comprising administering a secretion-inhibiting amount of at least one compound of the invention to a subject in need thereof.

In another embodiment, methods for treating and/or preventing bacterial infection, particularly Gram-negative bacterial infection, are provided comprising administering a therapeutically or prophylactically effective amount of at least one compound of the invention to a subject in need thereof.

These and other aspects of the invention will be more clearly understood with reference to the following preferred embodiments and detailed description.

### DETAILED DESCRIPTION OF THE INVENTION

Inhibition of Type III protein secretion is an important factor in the treatment and prevention of infection by Gram-negative bacteria. In accordance with the present invention, compounds that inhibit Type III protein secretion have been identified, and methods for their use provided.

A. Compounds of the Invention

In one aspect of the invention, compounds of the invention are provided which are useful in the inhibition of bacterial Type III protein secretion systems, and/or in the treatment or prevention of bacterial infection, particularly Gram-negative bacterial infection.

Where the compounds according to this invention have at least one stereogenic center, they may accordingly exist as enantiomers. Where the compounds possess two or more stereogenic centers, they may additionally exist as diastereomers. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Some of the compounds of the present invention may have trans and cis isomers. In addition, where the processes for the preparation of the compounds according to the invention give rise to a mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their component enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

Certain of the compounds of the invention, for example the imidazole derivatives, may exist as tautomers. It is understood that such tautomeric forms are intended to be encompassed within the scope of the invention.

As used herein, “enantiomerically pure” refers to compositions consisting substantially of a single isomer, preferably consisting of 90%, 92%, 95%, 98%, 99%, or 100% of a single isomer.

Included within the scope of the invention are the hydrated forms of the compounds that contain various amounts of water, for instance, the hydrate, hemihydrate, and sesquihydrate forms. The present invention also includes within its scope prodrugs and pharmaceutically acceptable salts of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985. Preferred compounds of the present invention useful in the inhibition of Type III protein secretion include those of Formula (I) as shown below. 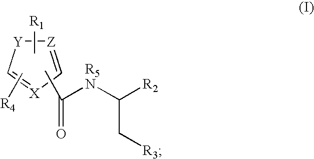
wherein X is N or CH; Y is O, S or NRₐ; and Z is CHₙ or N; and X, Y and Z taken together with the carbon atoms to which they are attached form a pyrazole, oxazole, triazole, thiazole, or imidazole ring;
Rₐ is hydrogen or R₄;
R₁ is aryl, substituted aryl, aryl-(C₂-C₄ alkynyl), heteroaryl, substituted heteroaryl, heterocyclyl, or substituted heterocyclyl;
R₂ is H or carboxy;
R₃ is aryl, optionally substituted by one or more halogen atoms; benzyloxy; benzylthio; benzylsulfinyl; or benzylsulfonyl; and R₄ is aryl or substituted aryl;
R₅ is hydrogen or lower alkyl;
when Rₐ is R₁ and Z is CHₙ, then n is 1, and when Rₐ is H, then Z is CHₙ and n is 0;
or an optical isomer, diastereomer or enantiomer thereof; or a pharmaceutically acceptable salt, hydrate, or prodrug thereof.

Unless otherwise noted, under standard nomenclature used throughout this disclosure the terminal portion of the designated side chain is described first, followed by the adjacent functionality toward the point of attachment.

Unless specified otherwise, the terms “alkyl,” “alkenyl,” and “alkynyl,” whether used alone or as part of a substituent group, include straight and branched chains having 1 to 8 carbon atoms, or any number within this range. The term “alkyl” refers to straight or branched chain hydrocarbons. “Alkenyl” refers to a straight or branched chain hydrocarbon with at least one carbon-carbon double bond. “Alkynyl” refers to a straight or branched chain hydrocarbon with at least one carbon-carbon triple bond. For example, alkyl radicals include methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl.

“Alkoxy” radicals are oxygen ethers formed from the previously described straight or branched chain alkyl groups.

The alkyl, alkenyl, alkynyl and alkoxy groups may be independently substituted with one or more members of the group including, but not limited to, halogen, alkyl, alkenyl, alkynyl, cycloalkyl, oxo, aryl, heteroaryl, heterocyclo, cyano, nitro, —OCOR₅, —OR₅, —SR₅, —SOR₅, —SO₂R₅, —COOR₅, —NR₅R₆, —CONR₅R₆, —OCONR₅R₆, —NHCOR₅, —NHCOOR₅, —NHC(NH)NHNO₂, and —NHCONR₅R₆, wherein R₅ and R₆ are independently selected from hydrogen, alkyl, alkenyl, alkynyl, cycloalkyl, aryl, heteroaryl, heterocyclo, aralkyl, heteroaralkyl, and heterocycloalkyl, or alternatively R₅ and R₆ may join to form a heterocyclic ring containing the nitrogen atom to which they are attached.

The term “acyl” as used herein, whether used alone or as part of a substituent group, means an organic radical having 2 to 6 carbon atoms (branched or straight chain) derived from an organic acid by removal of the hydroxyl group. The term “Ac” as used herein, whether used alone or as part of a substituent group, means acetyl.

The term “halo” or “halogen” means fluoro, chloro, bromo or iodo. Mono-, di-, tri-, and per-haloalkyl is an alkyl radical substituted by independent replacement of the hydrogen atoms thereon with halogen.

“Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 3 of the hydrogen atoms thereon with heterocyclyl, aryl, heteroaryl, halogen, OH, CN, mercapto, nitro, amino, C₁-C₈-alkyl, C₂-C₈-alkenyl, C₁-C₈-alkoxy, C₁-C₈-alkylthio, C₁-C₈-alkyl-amino, di (C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. “Ph” or “PH” denotes phenyl. “Bz” denotes benzoyl.

Whether used alone or as part of a substituent group, “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms. Exemplary heteroaryl groups include, for example, pyridinyl, pyrazinyl, pyrimidinyl, pyridazinyl, pyrrolyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, N-oxo-pyridyl, 1,1-dioxothienyl, benzothiazolyl, benzoxazolyl, benzothienyl, quinolinyl-N-oxide, benzimidazolyl, benzisothiazolyl, benzisoxazolyl, benzodiazinyl, benzofurazanyl, indazolyl, indolizinyl, benzofuryl, cinnolinyl, quinoxalinyl, pyrrolopyridinyl, furopyridinyl (such as furo[2,3-c]pyridinyl, furo[3,2-b]pyridinyl, or furo[2,3-b]pyridinyl), imidazo-pyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, and thienothienyl. The heteroaryl group may be substituted by independent replacement of 1 to 3 of the hydrogen atoms thereon with heterocyclyl, aryl, heteroaryl, halogen, OH, CN, mercapto, nitro, amino, C₁-C₈-alkyl, C₁-C₈-alkoxy, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Heteroaryl may be substituted with a mono-oxo to give for example a 4-oxo-1H-quinoline.

The terms “heterocycle,” “heterocyclic,” and “heterocyclyl” refer to an optionally substituted, fully saturated, partially saturated, or non-aromatic cyclic group which is, for example, a 4- to 7-membered monocyclic, 7- to 11-membered bicyclic, or 10- to 15-membered tricyclic ring system, which has at least one heteroatom in at least one carbon atom containing ring. Each ring of the heterocyclic group containing a heteroatom may have 1, 2, or 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom. The heterocyclic group may be substituted by independent replacement of 1 to 3 of the hydrogen atoms thereon with aryl, heteroaryl, halogen, C₁-C₈-alkyl, C₁-C₈-alkoxy, carboxy, alkoxycarbonyl, acyl, or carboxamide.

Exemplary monocyclic heterocyclic groups include pyrrolidinyl; oxetanyl; pyrazolinyl; imidazolinyl; imidazolidinyl; oxazolinyl; oxazolidinyl; isoxazolinyl; thiazolidinyl; isothiazolidinyl; tetrahydrofuryl; piperidinyl; piperazinyl; 2-oxopiperazinyl; 2-oxopiperidinyl; 2-oxopyrrolidinyl; 4-piperidinyl; tetrahydropyranyl; tetrahydrothiopyranyl; tetrahydrothiopyranyl sulfone; morpholinyl; thiomorpholinyl; thiomorpholinyl sulfoxide; thiomorpholinyl sulfone; 1,3-dioxolane; dioxanyl; thietanyl; thiiranyl; 2-oxazepinyl; azepinyl; and the like. Exemplary bicyclic heterocyclic groups include quinuclidinyl; tetrahydroisoquinolinyl; dihydroisoindolyl; dihydroquinazolinyl (such as 3,4-dihydro-4-oxo-quinazolinyl); dihydrobenzofuryl; dihydrobenzothienyl; benzothiopyranyl; dihydrobenzothiopyranyl; dihydrobenzothiopyranyl sulfone; benzopyranyl; dihydrobenzopyranyl; indolinyl; chromonyl; coumarinyl; isochromanyl; isoindolinyl; piperonyl; tetrahydroquinolinyl; and the like.

Substituted aryl, substituted heteroaryl, and substituted heterocycle may also be substituted with a second substituted aryl, a second substituted heteroaryl, or a second substituted heterocycle to give, for example, a 4-pyrazol-1-yl-phenyl or 4-pyridin-2-yl-phenyl.

The term “carbocyclic” refers to a saturated or unsaturated, non-aromatic, monocyclic, hydrocarbon ring of 3 to 7 carbon atoms.

Designated numbers of carbon atoms (e.g., C₁-C₈ or C₁₋₈) shall refer independently to the number of carbon atoms in an alkyl or cycloalkyl moiety or to the alkyl portion of a larger substituent in which alkyl appears as its prefix root.

The term “hydroxy protecting group” refers to groups known in the art for such purpose. Commonly used hydroxy protecting groups are disclosed, for example, in T. H. Greene and P. G. M. Wuts, Protective Groups in Organic Synthesis, 2nd edition, John Wiley & Sons, New York (1991), which is incorporated herein by reference. Illustrative hydroxyl protecting groups include but are not limited to tetrahydropyranyl; benzyl; methylthiomethyl; ethythiomethyl; pivaloyl; phenylsulfonyl; triphenylmethyl; trisubstituted silyl such as trimethylsilyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl; acyl and aroyl such as acetyl, benzoyl, pivaloylbenzoyl, 4-methoxybenzoyl, 4-nitrobenzoyl and phenylacetyl.

The phrase “a pharmaceutically acceptable salt” denotes one or more salts of the free base or free acid which possess the desired pharmacological activity of the free base or free acid as appropriate and which are neither biologically nor otherwise undesirable. These salts may be derived from inorganic or organic acids. Examples of inorganic acids are hydrochloric acid, nitric acid, hydrobromic acid, sulfuric acid, or phosphoric acid. Examples of organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, benzenesulfonic acid, salicylic acid and the like. Suitable salts are furthermore those of inorganic or organic bases, such as KOH, NaOH, Ca(OH)₂, Al(OH)₃, piperidine, morpholine, ethylamine, triethylamine and the like.

The term “subject” includes, without limitation, any animal or artificially modified animal. As a particular embodiment, the subject is a human.

The term “drug-resistant” or “drug-resistance” refers to the characteristics of a microbe to survive in the presence of a currently available antimicrobial agent such as an antibiotic at its routine, effective concentration.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein. Further, where a more generic substituent is set forth for any position in the molecules of the present invention, it is understood that the generic substituent may be replaced with more specific substituents, and the resulting molecules are within the scope of the molecules of the present invention.

B. Preparation of Compounds of the Invention

Compounds of the invention may be produced in any manner known in the art. By way of example, compounds of the invention may be prepared according to the following general schemes. The skilled artisan will also recognize the judicious choice of reactions, solvents, and temperatures are an important component in successful synthesis. While the determination of optimal conditions, etc. is routine, it will be understood that a variety of compounds can be generated in a similar fashion, using the guidance of the schemes below.

The starting materials used in preparing the compounds of the invention are known, made by published synthetic methods or available from commercial vendors.

It is recognized that the skilled artisan in the art of organic chemistry can readily carry out standard manipulations of the organic compounds without further direction; that is, it is well within the scope and practice of the skilled artisan to carry out such manipulations. These include, but are not limited to, reductions of carbonyl compounds to their corresponding alcohols, oxidations, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (Vol. 2), Feiser & Feiser, Reagents for Organic Synthesis (16 volumes), L. Paquette, Encyclopedia of Reagents for Organic Synthesis (8 volumes), Frost & Fleming, Comprehensive Organic Synthesis (9 volumes) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. Examples of these manipulations can be found for example in T. Greene, Protecting Groups in Organic Synthesis.

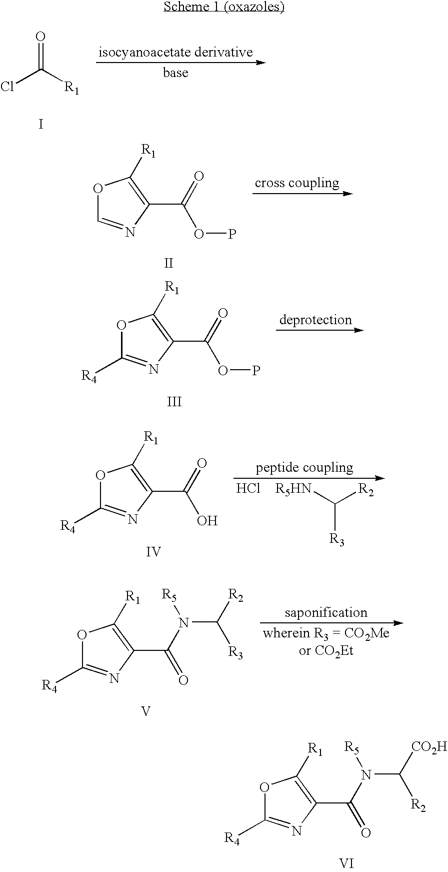

Oxazoles (VI) of Formula 1, wherein A and E are carbon, Z is CHₙ wherein n is 0, X is oxygen, and Y is NHₚ wherein p is 0, can be prepared by the method outlined in Scheme 1. Reaction of a suitably substituted acid chloride (I) with a suitable alkyl isocyanoacetate derivative, wherein P is an ester-based protecting group, in the presence of a tertiary amine base, such as DBU, triethylamine, diisopropylethyl-amine, or the like, in an inert solvent, such as methylene chloride, chloroform, tetrahydrofuran or acetonitrile, for from 1 to 48 hours at a temperature ranging from −20° C. to 37° C., affords the corresponding oxazole derivative (II). Cross-coupling reaction at the C-2 position of oxazole derivative (II) can be achieved by the following methods. Firstly, a palladium-catalyzed Negishi coupling reaction, where de-protonation at C-2 using n-butyl lithium followed by trans-metallation with zinc chloride at low temperature results in a zinc complex, which can then be coupled with an aryl iodide or bromide using bis(dibenzylideneacetone)palladium(0) (Pd(dba)₂) and tris(o-furyl)phosphine (TFP), triphenylphosphine (TPP) or 1,1-(diphenylphosphino)-ferrocene (dppf) as ligand. Secondly, a Heck-type cross-coupling reaction, catalyzed by, for example, palladium acetate, at C-2 with an aryl iodide or bromide using triphenylphosphine as ligand in the presence of copper (I) iodide and triethylamine. Removal of the ester protecting group of (III), for example by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester derivative, provides the corresponding acid derivative (IV). The conversion of acid (IV) to amide (V) can be carried out by reaction of (IV) with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (VI).

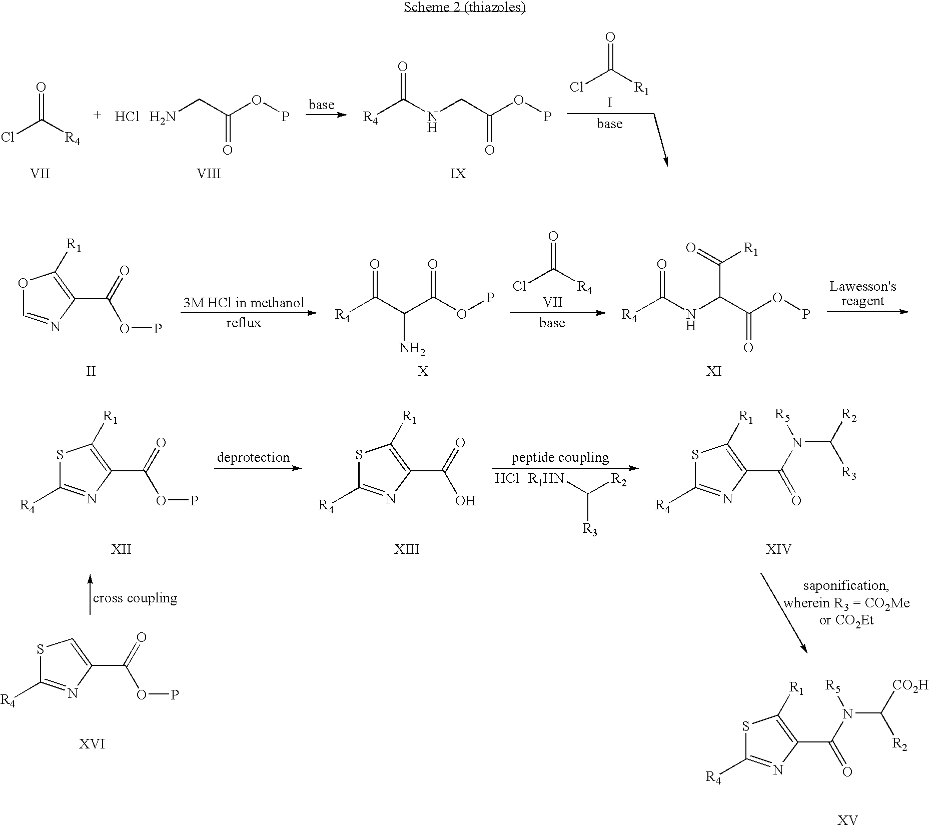

Thiazole compounds XV of Formula 1, wherein A and E are carbon, Z is CHₙ wherein n is 0, X is sulfur, and Y is NHₚ wherein p is 0, can be prepared by the method outlined in Scheme 2. Reaction of a suitably substituted acid chloride (VII) with a glycine ester hydrochloride derivative (VIII) wherein P is an ester-based protecting group, in the presence of a tertiary amine base, such as triethylamine, diisopropylethylamine, and the like, in an inert solvent, such as methylene chloride, chloroform, or tetrahydrofuran, for from 1 to 48 hours at a temperature ranging from—20° C. to 37° C., provides the corresponding amide derivative (IX). Conversion of 1× to XI can be achieved by treatment with a suitable base, such as NaH, LDA, sodium bis(trimethylsilyl)amide, or lithium bis(trimethylsilyl)amide, followed by a suitably substituted acid chloride (I), in a suitable inert anhydrous solvent, such as ethyl ether, THF, HMPA, or benzene, at a temperature ranging from −70° C. to 37° C. for from 1 to 48 hours. Alternatively, XI can be obtained from an oxazole derivative II by treatment with an acid, such as hydrochloric acid, or sulfuric acid, in an alcoholic solvent, such as methanol or ethanol, at a temperature ranging from 20° C. to 80° C. for 1 to 48 hours to afford X, followed by treatment of X with a suitably substituted acid chloride (VII) in the presence of a base, such as triethylamine, diisopropylethylamine, or the like, in a suitable inert solvent, such as methylene chloride, chloroform, or tetrahydrofuran, for from 1 to 48 hours at a temperature ranging from −20° C. to 37° C. Treatment of XI with Lawesson's reagent, in a suitable inert solvent, such as THF or dioxane, for from 1 to 24 hours at a temperature ranging from 20° C. to 110° C., affords the corresponding thiazole derivative (XII). Alternatively, XII may be obtained from 2,4-disubstituted thiazole derivative (XVI) via a Heck-type cross-coupling reaction, catalyzed by, for example, palladium acetate, at C-2 with an aryl iodide or bromide using triphenylphosphine as ligand in the presence of copper (I) iodide and triethylamine. Removal of the ester protecting group, for example by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester derivative, provides the corresponding acid derivative (XIII). The conversion of acid (XIII) to amide (XIV) can be carried out by reaction of XIII with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature form —20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (XV).

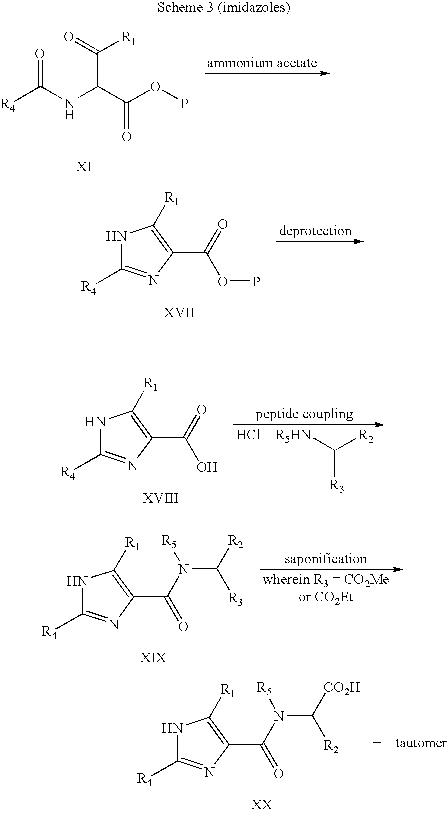

Analogous to the preparation of the thiazole derivative XV illustrated in Scheme 2, imidazole compounds XX of Formula 1, wherein A and E are carbon, Z is CHₙ wherein n is 0, and both X and Y are NHₚ wherein one p is 1 and the other p is 0, can be prepared by the method outlined in Scheme 3. Condensation of a suitably substituted ketoamide derivative XI with an ammonia equivalent, such as ammonium hydroxide or ammonium acetate, in the presence of an acid, such as acetic acid, in a suitable inert solvent, such as benzene, toluene, or xylene, for from 1 to 48 hours at a temperature ranging from 20° C. to 110° C., affords the corresponding imidazole derivative (XVII). Removal of the ester protecting group, for example by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester derivative, provides the corresponding acid derivative (XVIII). The conversion of acid (XVIII) to amide (XIX) can be carried out by reaction of XVIII with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or, an alcohol/water mixture, at a temperature ranging from 0° C. to 80° C. for from 1 to 48 hours, provides the corresponding acid derivative (XX).

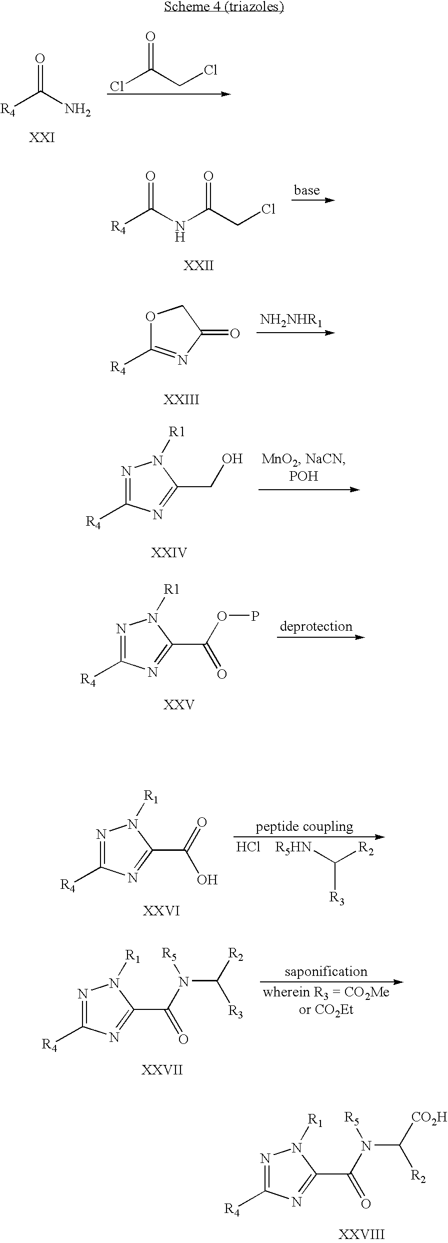

Triazole compounds (XXVIII) of Formula 1, wherein A and E are carbon, X and Y are NHₚ wherein p is 0, and Z is nitrogen, can be prepared by the method outlined in Scheme 4. Reaction of a suitably substituted amide (XXI) with chloroacetyl chloride in an inert solvent, such as benzene, toluene, or xylene, at a temperature ranging from 20° C. to 110° C., for from 1 to 48 hours, gives the corresponding imide derivative (XXII). Intramolecular alkylation of XXII, by treatment with a base, such as sodium t-butoxide, sodium hydride, or potassium hydride, in an inert solvent, such as THF, DMF, or DME, at a temperature ranging from 0° C. to 100° C., for from 1 to 24 hours, affords the corresponding oxazolone derivative XXIII. Reaction of XXIII with a suitably substituted hydrazine in an appropriate solvent such as methanol or ethanol at a temperature ranging from 20° C. to 80° C. for from 1 to 48 hours provides the corresponding triazole derivative (XXIV). Conversion of alcohol XXIV to ester XXV may be conducted in a single pot by combining triazole XXIV with an appropriate oxidizing agent, such as manganese dioxide, in the presence of a cyanating agent, such as sodium cyanide or potassium cyanide, and an alcohol nucleophile, such as methanol, ethanol, or the like, in an inert solvent, such as THF or DME. The reaction is carried out at a temperature ranging from 20° C. to 100° C. for from 1 to 48 hours. Hydrolysis of the ester protecting group of XXV, for example, by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester derivative, provides the corresponding acid derivative (XXVI). The conversion of acid (XXVI) to amide (XXVII) can be carried out by reaction of XXVI with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature form −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (XXVIII).

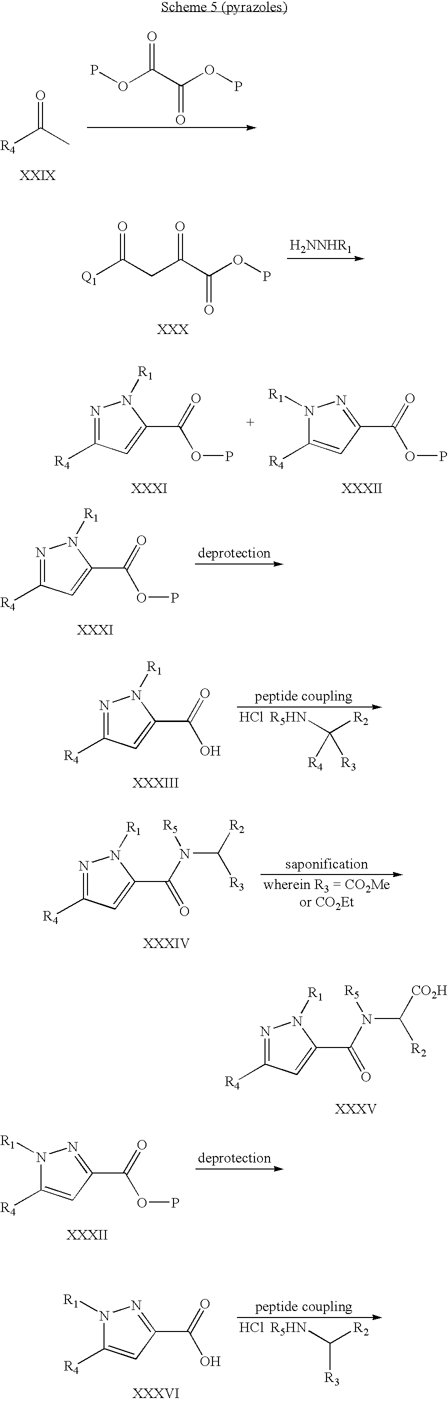

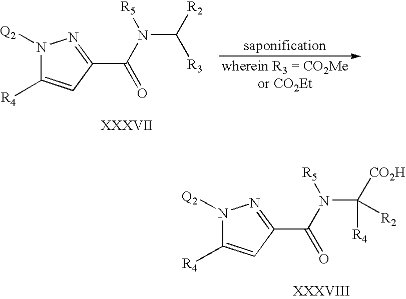

Pyrazole compounds (XXXV and XXXVIII) of Formula 1, wherein A and E are carbon, Y is CH, X is NHₚ wherein p is 0 and Z is N, can be prepared by the method outlined in Scheme 5. Reaction of a suitably substituted methyl ketone (XXIX) with a strong hindered base, such as lithium bis(trimethylsilyl)-amide, sodium bis(trimethylsilyl)amide or lithium diisopropylamide (LDA), in an inert solvent, such as tetrahydrofuran, ether or dichloromethane, at a temperature ranging from −78° C. to 0° C., for from 15 min to 2 hours, followed by treatment with a suitably substituted oxalate, at a temperature ranging from −78° C. to 25° C., for from 1 to 48 hours, gives the corresponding dioxo-butyric acid ester XXX. Conversion of XXX to the corresponding pyrazole derivative (XXXI or XXXII) may be accomplished by treatment with a suitably substituted hydrazine in a solvent, such acetic acid, at a temperature ranging from 20° C. to 120° C., for from 1 min to 48 hours. Hydrolysis of the ester protecting group of either XXXI or XXXII, for example, by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester derivative, provides the corresponding acid derivative (XXXIII or XXXVI). Conversion of the acid (XXXIII or XXXVI) to the corresponding amide (XXXIV or XXXVII) can be carried out by reaction of XXXIII or XXXVI with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (XXXV or XXXVIII).

In certain preferred embodiments, compounds of the invention may be resolved to enantiomerically pure compositions or synthesized as enantiomerically pure compositions using any method known in art. By way of example, compounds of the invention may be resolved by direct crystallization of enantiomer mixtures, by diastereomer salt formation of enantiomers, by the formation and separation of diastereomers or by enzymatic resolution of a racemic mixture.

These and other reaction methodologies may be useful in preparing the compounds of the invention, as recognized by one of skill in the art. Various modifications to the above schemes and procedures will be apparent to one of skill in the art, and the invention is not limited specifically by the method of preparing the compounds of the invention.

C. Methods of the Invention

In another aspect of the invention, methods are provided for the inhibition of Type III protein section, and/or the treatment and prevention of bacterial infection, particularly Gram-negative bacterial infection using the compounds described herein.

In one embodiment, the invention is directed to methods for inhibiting Type III protein secretion comprising administering a secretion-inhibiting amount of at least one compound of the invention to a subject in need thereof.

In yet another embodiment, methods for treating or prevention of bacterial infection, particularly Gram-Negative bacterial infection are provided comprising administering a therapeutically or prophylactically effective amount of at least one compound of the invention to a subject in need thereof.

According to the methods of the invention, the compound(s) may be administered to the subject via any drug delivery route known in the art. Specific exemplary administration routes include oral, ocular, rectal, buccal, topical, nasal, ophthalmic, subcutaneous, intramuscular, intravenous (bolus and infusion), intracerebral, transdermal, and pulmonary.

The terms “secretion-inhibiting amount”, “therapeutically effective amount”, and “prophylactically effective amount”, as used herein, refer to an amount of a compound of the invention sufficient to treat, ameliorate, or prevent the identified disease or condition, or to exhibit a detectable therapeutic, prophylactic, or inhibitory effect. The effect can be detected by, for example, the assays disclosed in the following examples. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

For any compound, the therapeutically or prophylactically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

More specifically, the concentration-biological effect relationships observed with regard to the compound(s) of the present invention indicate an initial target plasma concentration ranging from approximately 5 μg/mL to approximately 100 μg/mL, preferably from approximately 10 μg/mL to approximately 100 μg/mL, more preferably from approximately 20 μg/mL to approximately 100 μg/mL. To achieve such plasma concentrations, the compounds of the invention may be administered at doses that vary from 0.1 μg to 100,000 mg, depending upon the route of administration. Guidance as to particular dosages and methods of delivery is provided in the literature and is generally available to practitioners in the art. In general the dose will be in the range of about 1 mg/day to about 10 g/day, or about 0.1 g to about 3 g/day, or about 0.3 g to about 3 g/day, or about 0.5 g to about 2 g/day, in single, divided, or continuous doses for a patient weighing between about 40 to about 100 kg (which dose may be adjusted for patients above or below this weight range, particularly children under 40 kg).

The exact dosage will be determined by the practitioner, in light of factors related to the subject that requires treatment. Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

D. Metabolites of the Compounds of the Invention

Also falling within the scope of the present invention are the in vivo metabolic products of the compounds described herein. Such products may result for example from the oxidation, reduction, hydrolysis, amidation, esterification and the like of the administered compound, primarily due to enzymatic processes. Accordingly, the invention includes compounds produced by a process comprising contacting a compound of this invention with a mammalian tissue or a mammal for a period of time sufficient to yield a metabolic product thereof. Such products typically are identified by preparing a radio-labeled (e.g. ¹⁴C or ³H) compound of the invention, administering it in a detectable dose (e.g., greater than about 0.5 mg/kg) to a mammal such as rat, mouse, guinea pig, monkey, or to man, allowing sufficient time for metabolism to occur (typically about 30 seconds to 30 hours), and isolating its conversion products from urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds of the invention even if they possess no biological activity of their own.

E. Pharmaceutical Compositions of the Invention

While it is possible for the compounds of the present invention to be administered neat, it may be preferable to formulate the compounds as pharmaceutical compositions. As such, in yet another aspect of the invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pbarmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

More particularly, the pharmaceutical compositions of the invention comprise a therapeutically or prophylactically effective amount of at least one compound of the present invention, together with one or more pharmaceutically acceptable excipients. Optionally, the pharmaceutical compositions of the invention may comprise a combination of compounds of the present invention, or may include a second active ingredient useful in the treatment or prevention of bacterial infection (e.g., anti-bacterial or anti-microbial agents).

Formulations of the present invention, e.g., for parenteral or oral administration, are most typically solids, liquid solutions, emulsions or suspensions, while inhalable formulations for pulmonary administration are generally liquids or powders, with powder formulations being generally preferred. A preferred pharmaceutical composition of the invention may also be formulated as a lyophilized solid that is reconstituted with a physiologically compatible solvent prior to administration. Alternative pharmaceutical compositions of the invention may be formulated as syrups, creams, ointments, tablets, and the like.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exist a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions of the invention may be in the form of a sterile injectable preparation, such as a sterile injectable aqueous emulsion or oleaginous suspension. This emulsion or suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,2-propane-diol. The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables.

Generally, the compounds of the present invention useful in the methods of the present invention are substantially insoluble in water and are sparingly soluble in most pharmaceutically acceptable protic solvents and in vegetable oils. However, the compounds are generally soluble in medium chain fatty acids (e.g., caprylic and capric acids) or triglycerides and have high solubility in propylene glycol esters of medium chain fatty acids. Also contemplated in the invention are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In a preferred embodiment, the compounds of the present invention may be formulated for oral administration in a lipid-based formulation suitable for low solubility compounds. Lipid-based formulations can generally enhance the oral bioavailability of such compounds. As such, a preferred pharmaceutical composition of the invention comprises a therapeutically or prophylactically effective amount of a compound of the present invention, together with at least one pharmaceutically acceptable excipient selected from the group consisting of: medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In an alternative preferred embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-β-cyclodextrin (HPBC), which may be added to any of the above-described compositions to further improve the aqueous solubility characteristics of the compounds of the present invention. In one embodiment, the composition comprises 0.1% to 20% hydroxypropyl-β-cyclodextrin, more preferably 1% to 15% hydroxypropyl-β-cyclodextrin, and even more preferably from 2.5% to 10% hydroxypropyl-β-cyclodextrin. The amount of solubility enhancer employed will depend on the amount of the compound of the present invention in the composition.

F. Combination Therapy

It is also possible to combine any compound of the present invention with one or more other active ingredients useful in the treatment or prevention of bacterial infection, including compounds, in a unitary dosage forms, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

The skilled artisan will recognize that a variety of active ingredients may be administered in combination with the compounds of the present invention that may act to augment or synergistically enhance the Type III protein secretion-inhibiting activity of the compounds of the invention.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of certain compounds of the invention, and the testing of these compounds in vitro and/or in vivo. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

The following examples describe in detail the chemical synthesis of representative compounds of the present invention. The procedures are illustrations, and the invention should not be construed as being limited by the chemical reactions and conditions they express. No attempt has been made to optimize the yields obtained in these reactions, and it would be obvious to one skilled in the art that variations in reaction times, temperatures, solvents, and/or reagents could increase the yields.

### Example 1

### Preparation of Compounds of the Invention

Compounds of Formula I may be prepared according to the schemes disclosed herein as follows.

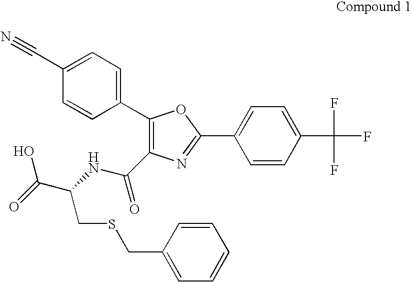

### (R)-3-Benzylsulfanyl-2-{[5-(4-cyanophenyl)-2-(4-trifluoromethylphenyl)oxazole-4-carbonyl]amino}propionic acid

### Step A: 5-(4-Cyanophenyl)oxazole-4-carboxylic acid methyl ester

To a solution of methyl isocyanoacetate (1.82 mL, 20 mmol) and 4-cyano-benzoyl chloride (3.31 g, 20 mmol) in THF (20.0 mL) at 0° C. was added triethylamine (8.4 mL, 60 mmol) and the resulting reaction mixture was stirred at 0° C. for 30 min and at room temperature for an additional 8 h. The crude reaction mixture was concentrated in vacuo and the solid was dissolved in ethyl acetate (30 mL) and washed with water (30 mL×2). The organic layer was separated, dried over sodium sulfate and the filtrate was concentrated in vacuo. Purification by medium pressure liquid chromatography on silica gel (1:4 ethyl acetate/hexanes) gave 4.1 g (90%) of the title compound. MS 229.1 (M+H)⁺.

### Step B: 5-(4-Cyanophenyl)-2-(4-trifluoromethylphenyl)oxazole-4-carboxylic acid methyl ester

A mixture of 5-(4-cyanophenyl)oxazole-4-carboxylic acid methyl ester from Step A (1.14 g, 5.0 mmol), 1-iodo-4-trifluoromethylbenzene (0.96 mL, 6.5 mmol), palladium acetate (0.11 g, 0.5 mmol), triphenylphosphine (0.26 g, 1.0 mmol), copper iodide (I) (1.24 g, 6.5 mmol) and triethylamine (0.90 mL, 6.5 mmol) in acetonitrile (25.0 mL) was heated at reflux temperature for 12 h. The reaction mixture was then cooled to room temperature, filtered through a pad of Celite and the filtrate was concentrated in vacuo. Purification by medium pressure liquid chromatography on silica gel (1:4 ethyl acetate/hexanes) gave 1.15 g (62%) of the title compound. MS 373.1 (M+H)⁺.

### Step C: 5-(4-Cyanophenyl)-2-(4-trifluoromethylphenyl)oxazole-4-carboxylic acid

To a solution of 5-(4-cyanophenyl)-2-(4-trifluoromethylphenyl)oxazole-4-carboxylic acid methyl ester from Step B (186 mg, 0.5 mmol) in THF (1.0 mL) was added 1N lithium hydroxide (aq., 1.0 mL) and the reaction mixture was stirred at room temperature for 3 h. After acidification with 1N HCl to pH 4-5, the mixture was diluted with ethyl acetate and washed with water. The organic layer was separated, dried over sodium sulfate and the filtrate was concentrated in vacuo giving 154 mg (86%) of the title compound, which was used without further purification. MS 359.0 (M+H)⁺.

### Step D: 3-Benzylsulfanyl-2-{[5-(4-cyanophenyl)-2-(4-trifluoromethylphenyl)oxazole-4-carbonyl]amino}propionic acid methyl ester

A mixture of 5-(4-cyanophenyl)-2-(4-trifluoromethylphenyl)oxazole-4-carboxylic acid from Step C (100 mg, 0.28 mmol), H-Cys(Bzl)-L-OMe hydrochloride (110 mg, 0.42 mmol), PyBrop (196 mg, 0.42 mmol), 4-dimethylaminopyridine (51 mg, 0.42 mmol) and triethylamine (0.059 mL, 0.42 mmol) in dichloromethane (5.0 mL) was stirred at room temperature for 12 h. The crude mixture was washed with water and concentrated in vacuo. Purification by medium pressure liquid chromatography on silica gel (1:9 ethyl acetate/hexanes) gave 86 mg (54%) of the title compound. MS 566.1 (M+H)⁺.

### Step E (R)-3-Benzylsulfanyl-2-{[5-(4-cyanophenyl)-2-(4-trifluoromethylphenyl)oxazole-4-carbonyl]amino}propionic acid

To a solution of 3-benzylsulfanyl-2-{[5-(4-cyanophenyl)-2-(4-trifluoro-methylphenyl)oxazole-4-carbonyl]amino}propionic acid methyl ester from Step D (85 mg, 0.15 mmol) in THF (1.0 mL) was added 1N lithium hydroxide (aq., 1.0 mL) and the reaction mixture was stirred at room temperature for 3 h. After acidification with 1N HCl to pH 4-5, the white precipitate was collected by filtration giving 80 mg (97%) of the title compound. MS 552.0 (M+H)⁺.

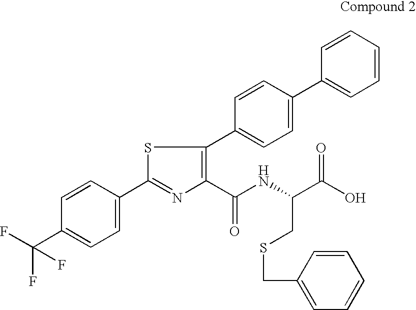

### (R)-3-Benzylsulfanyl-2-{[5-biphenyl-4-yl-2-(4-trifluoromethylphenyl)thiazole-4-carbonyl]amino}propionic acid

### Step A: 5-Biphenyl-4-yl-oxazole-4-carboxylic acid methyl ester

Methyl isocyanoacetate (2.0 g, 20.2 mmol), 4-phenylbenzoyl chloride (4.37 g, 20.2 mmol) and triethylamine (8.4 ml, 60.6 mmol) were dissolved in THF (20 ml) under N₂. The reaction mixture was stirred at room temperature for 18 hours and then heated at 60° C. 3 hours. The solvent was evaporated. The resulting crude solid was washed with water (5 ml) and hexane (30 ml) then crystallized in methanol to give the title compound (4.23 g, 15.15 mmol, 75%). MS 280.1 (M+H)⁺.

### Step B: 2-Amino-3-biphenyl-4-yl-3-oxo-propionic acid methyl ester hydrochloride.

5-Biphenyl-4-yl-oxazole-4-carboxylic acid methyl ester from Step A (1.0 g, 3.58 mmol) was dissolved in 20 ml methanol and dichloromethane (8 ml). Concentrated HCl (35%, 7 ml) was added, and the reaction mixture was heated at 50° C. under N₂ for 15 hours. The solvent was evaporated. The resulting residue was partitioned between water (pH 1) and EtOAc. The organic layer was discarded. The pH of the water layer was adjusted to ˜10 by addition of dilute sodium hydroxide and extracted with ethyl acetate. The organic layer was dried, the solvent removed in vacuo, and the residue recrystallized from methanol and EtOAc to give the title compound (0.738 g, 90% purity, 67% yield). MS 270.1 (M+H)⁺.

### Step C: 3-Biphenyl-4-yl-3-oxo-2-(4-trifluoromethyl-benzoylamino)-propionic acid methyl ester

2-Amino-3-biphenyl-4-yl-3-oxo-propionic acid methyl ester hydrochloride from Step B (200 mg, 0.65 mmol), 4-trifluoromethylbenzoyl chloride (0.164 mmol) and triethylamine (0.328 mmol) were put into anhydrous THF (2 ml). The reaction mixture was stirred at room temperature under N₂ for 1 hour. The solvent was evaporated. The resulting residue was partitioned between water and EtOAc. The organic layer was dried over MgSO₄, filtered and concentrated. The crude product was subjected to MPLC to give the title compound (200 mg, 0.454 mmol, 70% yield). MS 464.1 (M+Na)⁺.

### Step D: 5-Biphenyl-4-yl-2-(4-trifluoromethylphenyl)thiazole-4-carboxylic acid methyl ester

3-Biphenyl-4-yl-3-oxo-2-(4-trifluoromethylbenzoylamino)propionic acid methyl ester from Step C (47 mg, 0.107 mmol) and Lawesson's reagent (43 mg, 0.107 mmol) were dissolved in THF (anhydrous, 2 ml). The reaction mixture was refluxed under N₂ for 2 hours. The solvent was evaporated. The resulting residue was subjected to MPLC to give the title compound (38 mg, 0.087 mmol, 81% yield) as a white solid. MS 440.1 (M+H)⁺.

### Step E: 5-Biphenyl-4-yl-2-(4-trifluoromethylphenyl)thiazole-4-carboxylic acid.

5-Biphenyl-4-yl-2-(4-trifluoromethylphenyl)thiazole-4-carboxylic acid methyl ester from Step D (38 mg, 0.087 mmol) was dissolved in THF (2 ml) NaOH solution (1 N, 1 ml) was added. The reaction mixture was stirred at room temperature for 5 hours. The THF was evaporated. The resulting water solution was acidified to pH 1 with HCl (1 N). A white precipitate formed, and was filtered to give the title compound as a crude product, which was used without further purification.

### Step F: 3-Benzylsulfanyl-2-{[5-biphenyl-4-yl-2-(4-trifluoromethylphenyl)thiazole-4-carbonyl]amino}propionic acid methyl ester

5-Biphenyl-4-yl-2-(4-trifluoromethylphenyl)thiazole-4-carboxylic acid from Step D (52 mg, crude, 0.122 mmol), (L)-S-benzylcysteine methyl ester hydrochloride (32 mg, 0.122 mmol), PyBop (70 mg, 0.144 mmol) and triethylamine (0.13 ml, 0.268 mmol) were dissolved in CH₂Cl₂ (3 ml). The mixture was stirred at room temperature for 3 hours. The mixture was concentrated, and subject to MPLC to give the title compound. MS 633.1 (M+H)⁺.

### Step G: (R)-3-Benzylsulfanyl-2-{[5-biphenyl-4-yl-2-(4-trifluoromethylphenyl)thiazole-4-carbonyl]amino}propionic acid

3-Benzylsulfanyl-2-{[5-biphenyl-4-yl-2-(4-trifluoromethylphenyl)thiazole-4-carbonyl]amino}propionic acid methyl ester was dissolved in THF (2 ml) and NaOH solution (1 N, 1 ml) was added. The reaction mixture was stirred at room temperature for hours. The THF was evaporated. The resulting water solution was acidified to pH 1 with HCl (1 N). A white precipitate formed, and was filtered to give the title compound (15 mg, 0.024 mmol, 28% yield over Steps E-G). MS 619.0 (M+H)⁺.

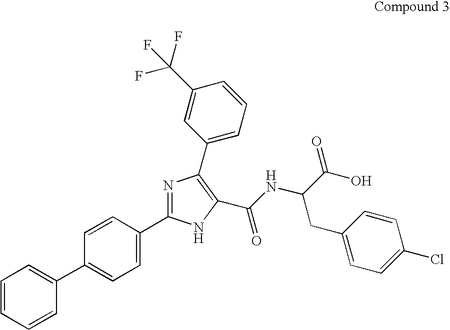

### 2-{[2-Biphenyl-4-yl-5-(3-trifluoromethylphenyl)-3H-imidazole-4-carbonyl]amino}-3-(4-chlorophenyl)propionic acid

### Step A: [(Biphenyl-4-carbonyl)amino]acetic acid methyl ester

To a mixture of glycine methyl ester (1.25 g, 10 mmol) and biphenyl-4-carbonyl chloride (2.16 g, 10 mmol) in dichloromethane (20 mL) was added dropwise triethylamine (2.1 mL, 15 mmol). The resulting reaction mixture was stirred at room temperature for 4 h. After washing with water, aqueous sodium carbonate and 1N hydrochloric acid, the organic layer was separated, dried over sodium sulfate and filtered. The filtrate was concentrated in vacuo to give 1.3 g of the title compound (50%). MS 270.0 (M+H)⁺.

### Step B: 2-[(Biphenyl-4-carbonyl)amino]-3-oxo-3-(3-trifluoromethylphenyl)propionic acid methyl ester

To a solution of [(biphenyl-4-carbonyl)-amino]acetic acid methyl ester from Step A (269 mg, 1 mmol) in THF/HMPA (3.0 mL/3.0 mL) at −78° C. under a nitrogen atmosphere was added lithium diisopropylamide (LDA, 1.7 mL of 1.5 M solution in hexane) and the resulting reaction mixture was stirred at −78° C. for 30 min. 3-Trifluoromethylbenzoyl chloride (0.15 mL, 1 mmol) was added slowly to the reaction mixture and it was then stirred at −78° C. for an additional 1 h followed by stirring at room temperature for 4 h. Saturated ammonium chloride (aq.) was added to the reaction mixture and the organic layer was diluted with ethyl acetate. After separation the organic layer was dried over sodium sulfate, filtered, and the filtrate was concentrated in vacuo to give the crude product. Purification by medium pressure liquid chromatography on silica gel (1:9 ethyl acetate/hexanes) gave 170 mg (39%) of the title compound. MS 442.1 (M+H)⁺.

### Step C: 2-Biphenyl-4-yl-5-(3-trifluoromethylphenyl)-3H-imidazole-4-carboxylic acid methyl ester (both tautomers are included)

A mixture of 2-[(biphenyl-4-carbonyl)-amino]-3-oxo-3-(3-trifluoromethyl-phenyl)propionic acid methyl ester from Step B (170 mg, 0.39 mmol), ammonium acetate (178 mg, 2.3 mmol) and acetic acid (0.5 mL) in o-xylene (5.0 mL) was heated at reflux temperature for 16 h. The reaction mixture was then cooled to room temperature and concentrated in vacuo. The crude material was then dissolved in ethyl acetate (15 mL) and washed with saturated sodium bicarbonate (aq.) and water. After separation the organic layer was dried over sodium sulfate, filtered, and the filtrate was concentrated in vacuo to give the crude product. Purification by medium pressure liquid chromatography on silica gel (1:9 ethyl acetate/hexanes) gave 98 mg (59%) of the title compound. MS 423.1 (M+H)⁺.

### Step D: 2-{[2-Biphenyl-4-yl-5-(3-trifluoromethylphenyl)-3H-imidazole-4-carbonyl]amino}-3-(4-chlorophenyl)propionic acid

The title compound was prepared by a procedure analogous to that of Step C-E of Compound 1 by substituting 2-biphenyl-4-yl-5-(3-trifluoromethyl-phenyl)-3H-imidazole-4-carboxylic acid methyl ester from Step C of Compound 3 for the 5-(4-cyanophenyl)-2-(4-trifluoromethylphenyl)oxazole-4-carboxylic acid methyl ester of Step C of Compound 1; and by substituting D,L-4-chlorophenylalanine ethyl ester hydrochloride for the H-Cys(Bzl)-L-OMe hydrochloride of Step D of Compound 1. MS 588.2 (M−H)⁻.

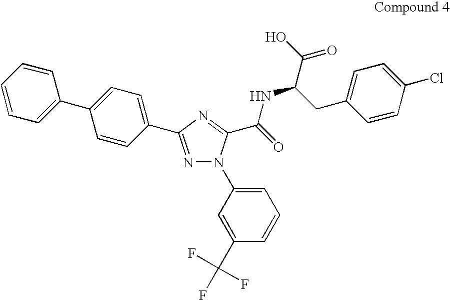

### (R)-2-{[5-Biphenyl-4-yl-2-(3-trifluoromethylphenyl)-2H-[1,2,4]triazole-3-carbonyl]amino}-3-(4-chlorophenyl)propionic acid

### Step A: Biphenyl-4-carboxylic acid (2-chloroacetyl)amide

A suspension of biphenyl-4-carboxylic acid amide (1.97 g, 10 mmol) and chloroacetyl chloride (1.2 mL, 15 mmol) in toluene (20 mL) was heated at reflux temperature for 1.5 h. It was then cooled to room temperature and the resulting precipitate was filtered and washed with hexanes to give 1.64 g of the title compound (60%). MS 274.1 (M+H)⁺.

### Step B: 2-Biphenyl-4-yl-oxazol-4-one

To a suspension of NaH (95% tech., 25 mg, 1 mmol) in DME (20 mL) was added biphenyl-4-carboxylic acid (2-chloroacetyl)amide from Step A (273 mg, 1 mmol), and the reaction mixture was stirred at room temperature for 30 min. It was then heated at reflux temperature for 2-8 h. After the reaction mixture was cooled to room temperature, a small amount of precipitate was removed by filtering through a pad of Celite and the filtrate was concentrated in vacuo to give the crude product. Purification by medium pressure liquid chromatography on silica gel (1:9 ethyl acetate/hexanes) gave 90 mg (38%) of the title compound. MS 238.1 (M+H)⁺.

### Step C: [5-Biphenyl-4-yl-2-(3-trifluoromethylphenyl)-2H-[1,2,4]triazol-3-yl]methanol

A mixture of 2-biphenyl-4-yl-oxazol-4-one from Step B (88 mg, 0.37 mmol) and (3-trifluoromethylphenyl)hydrazine (0.048 mL, 0.37 mmol) in ethanol (5.0 mL) was heated at reflux temperature for 30 min. The reaction mixture was cooled to room temperature and concentrated in vacuo. Purification of the crude product by medium pressure liquid chromatography on silica gel (1:3 ethyl acetate/hexanes) gave 90 mg (62%) of the title compound. MS 396.1 (M+H)⁺.

### Step D: 5-Biphenyl-4-yl-2-(3-trifluoromethylphenyl)-2H-[1,2,4]triazole-3-carboxylic acid methyl ester

To a mixture of [5-biphenyl-4-yl-2-(3-trifluoromethylphenyl)-2H-[1,2,4]triazol-3-yl]methanol from Step C (99 mg, 0.25 mmol), sodium cyanide (12 mg, 0.25 mmol) and activated manganese dioxide (348 mg, 4 mmol) in THF (10 mL) was added methanol (0.05 mL, 1.3 mmol). The reaction mixture was heated at reflux temperature for 16 h and then cooled to room temperature. After passing through a pad of Celite, the filtrate was concentrated in vacuo to give the crude product. Purification of the crude product by medium pressure liquid chromatography on silica gel (1:4 ethyl acetate/hexanes) gave 42 mg (40%) of the title compound. MS 424.1 (M+H)⁺.

### Step E: (R)-2-{[5-Biphenyl-4-yl-2-(3-trifluoromethylphenyl)-2H-[1,2,4]triazole-3-carbonyl]amino}-3-(4-chlorophenyl)propionic acid

The title compound was prepared by a procedure analogous to that of Steps C-E of Compound 1 by substituting 5-biphenyl-4-yl-2-(3-trifluoromethylphenyl)-2H-[1,2,4]triazole-3-carboxylic acid methyl ester from Step D of Compound 4 for the 5-(4-cyanophenyl)-2-(4-trifluoromethylphenyl)oxazole-4-carboxylic acid methyl ester of Step C of Compound 1; and by substituting D-4-chlorophenylalanine methyl ester hydrochloride for the H-Cys(Bzl)-L-OMe hydrochloride of Step D of Compound 1. MS 591.0 (M+H)⁺.

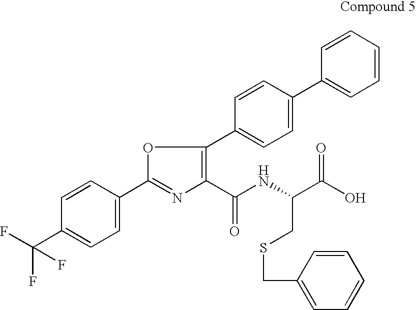

### (R)-3-Benzylsulfanyl-2-{[5-biphenyl-4-yl-2-(4-trifluoromethylphenyl)oxazole-4-carbonyl]amino}propionic acid

The title compound was prepared by a procedure analogous to that of Compound 1 by substituting biphenyl-4-carbonyl chloride for the 4-cyanobenzoyl chloride of Step A of Compound 1. MS 601.0 (M−H)⁻.

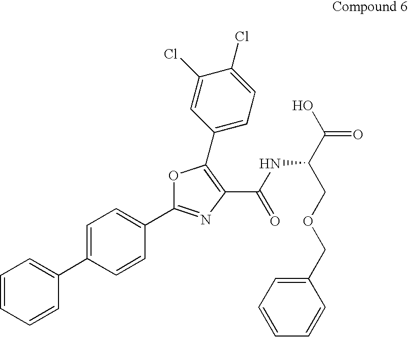

### (S)-3-Benzyloxy-2-{[2-biphenyl-4-yl-5-(3,4-dichlorophenyl)oxazole-4-carbonyl]amino}propionic acid

The title compound was prepared by a procedure analogous to that of Compound 1 by substituting 3,4-dichlorobenzoyl chloride for the 4-cyanobenzoyl chloride of Step A of Compound 1; and by substituting 4-bromobiphenyl for the 1-iodo-4-trifluoromethylbenzene of Step B of Compound 1; and by substituting H-Ser(Bzl)-L-OMe hydrochloride for the H-Cys(Bzl)-L-OMe hydrochloride of Step D of Compound 1. MS 585.0 (M−H)⁻.

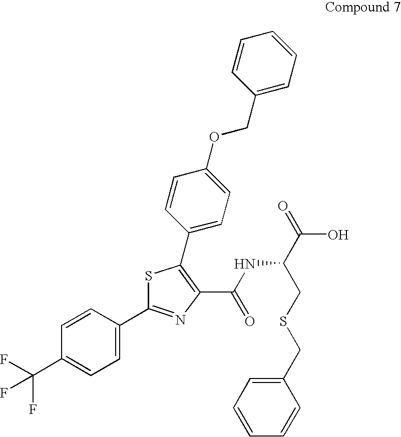

### (R)-2-{[5-(4-Benzyloxyphenyl)-2-(4-trifluoromethylphenyl)thiazole-4-carbonyl]amino}-3-benzylsulfanylpropionic acid

### Step A: 5-(4-Benzyloxyphenyl)-2-(4-trifluoromethylphenyl)thiazole-4-carboxylic acid ethyl ester

A mixture of 2-(4-trifluoromethylphenyl)thiazole-4-carboxylic acid ethyl ester (commercially available, 301 mg, 1.0 mmol), 4-benzyloxy-1-iodobenzene (620 mg, 2.0 mmol), palladium acetate (22 mg, 0.1 mmol), copper (I) iodide (381 mg, 2.0 mmol), triphenylphosphine (52 mg, 0.2 mmol) and triethylamine (0.28 mL, 2.0 mmol) in dimethyl formamide (3.0 mL) was heated at 130° C. for 12 h. It was then cooled to room temperature and diluted with ethyl acetate (15 mL). After washing with water multiple times, the organic layer was separated, dried over sodium sulfate and filtered. The filtrate was concentrated in vacuo to give the crude product. Purification of the crude product by medium pressure liquid chromatography on silica gel (1:9 ethyl acetate/hexanes) gave 85 mg (18%) of the title compound. MS 484.1 (M+H)⁺.

### Step B: (R)-2-{[5-(4-Benzyloxyphenyl)-2-(4-trifluoromethylphenyl)thiazole-4-carbonyl]amino}-3-benzylsulfanylpropionic acid

The title compound was prepared by a procedure analogous to that of Steps C-E of Compound 1 by substituting 5-(4-benzyloxyphenyl)-2-(4-trifluoromethylphenyl)-thiazole-4-carboxylic acid ethyl ester from Step A of Compound 7 for the 5-(4-cyanophenyl)-2-(4-trifluoromethylphenyl)oxazole-4-carboxylic acid methyl ester of Step C of Compound 1. MS 647.0 (M−H)⁻.

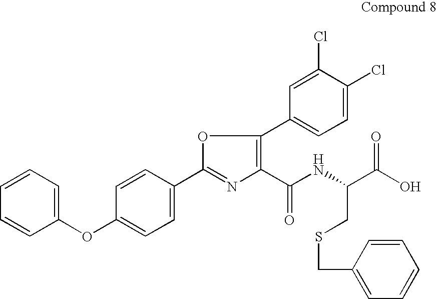

### (R)-3-Benzylsulfanyl-2-{[5-(3,4-dichlorophenyl)-2-(4-phenoxyphenyl)oxazole-4-carbonyl]amino}propionic acid

The title compound was prepared by a procedure analogous to that of Compound 1 by substituting 3,4-dichlorobenzoyl chloride for the 4-cyanobenzoyl chloride of Step A of Compound 1; and by substituting 1-iodo-4-phenoxybenzene for the 1-iodo-4-trifluoromethylbenzene of Step B of Compound 1. MS 618.8 (M−H)⁻.

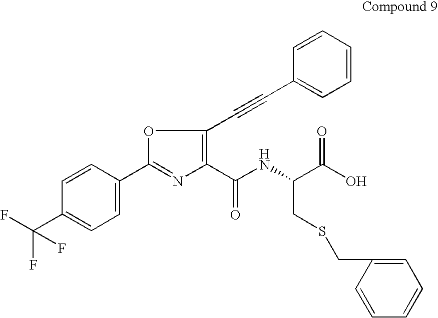

### (R)-3-Benzylsulfanyl-2-{[5-phenylethynyl-2-(4-trifluoromethylphenyl)oxazole-4-carbonyl]amino}propionic acid

The title compound was prepared by a procedure analogous to that of Compound 1 by substituting phenyl-propynoyl chloride for the 4-cyanobenzoyl chloride of Step A of Compound 1. MS 549.2 (M−H)⁻.

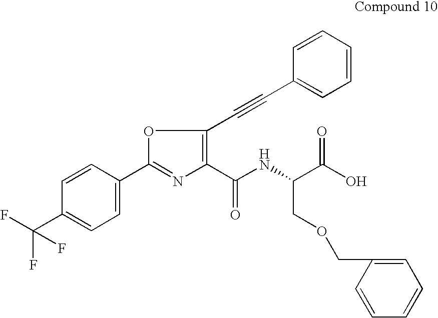

### (S)-3-Benzyloxy-2-{[5-phenylethynyl-2-(4-trifluoromethylphenyl)oxazole-4-carbonyl]amino}propionic acid

The title compound was prepared by a procedure analogous to that of Compound 1 by substituting phenylpropynoyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 533.1 (M−H)⁻.

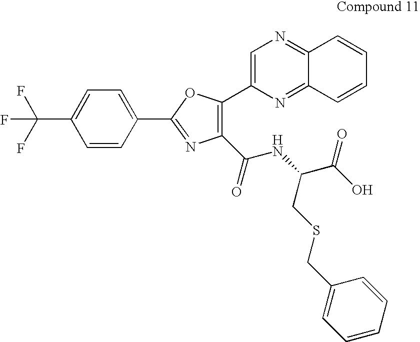

### (R)-3-Benzylsulfanyl-2-{[5-quinoxalin-2-yl-2-(4-trifluoromethylphenyl)oxazole-4-carbonyl]amino}propionic acid

The title compound was prepared by a procedure analogous to that of Compound 1 by substituting quinoxaline-2-carbonyl chloride for the 4-cyanobenzoyl chloride of Step A of Compound 1. MS 577.0 (M−H)⁻.

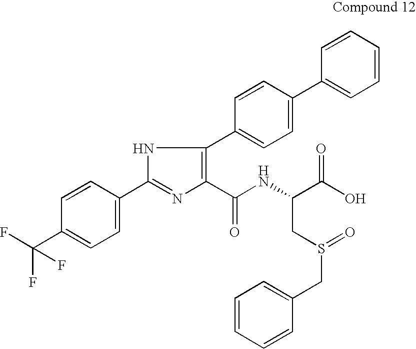

### (R)-2-{[5-Biphenyl-4-yl-2-(4-trifluoromethylphenyl)-1H-imidazole-4-carbonyl]amino}-3-phenylmethanesulfinylpropionic acid

### Step A: 5-Biphenyl-4-yl-oxazole-4-carboxylic acid methyl ester

Methyl isocyanoacetate (2.0 g, 20.2 mmol), 4-phenylbenzoyl chloride (4.37 g, 20.2 mmol) and triethylamine (8.4 ml, 60.6 mmol) were dissolved in THF (20 ml) under N₂. The reaction mixture was stirred at room temperature for 18 hours, then heated to 60° C. for 3 hours. The solvent was evaporated. The resulting crude solid was washed with water (5 ml) and hexane (30 ml), then crystallized in methanol to give the title compound (4.23 g, 15.15 mmol, 75%). MS 280.1 (M+H)⁺.

### Step B: 2-Amino-3-biphenyl-4-yl-3-oxopropionic acid methyl ester hydrochloride.

5-Biphenyl-4-yl-oxazole-4-carboxylic acid methyl ester from Step A (1.0 g, 3.58 mmol) was dissolved in 20 ml methanol and dichloromethane (8 ml). Concentrated HCl (35%, 7 ml) was added, and the reaction mixture was heated at 50° C. under N₂ for 15 hours. The solvent was evaporated. The resulting residue was dissolved in dilute HCl (pH 1) and extracted with EtOAc. The organic layer was discarded. The aqueous layer was co-evaporated with methanol in vacuo, and the residue was then recrystallized from methanol and EtOAc to give the title compound (0.738 g, 90% purity, 67% yield). MS 270.1 (M+H)⁺.

### Step C: 3-Biphenyl-4-yl-3-oxo-2-(4-trifluoromethylbenzoylamino)propionic acid methyl ester

2-Amino-3-biphenyl-4-yl-3-oxo-propionic acid methyl ester hydrochloride from Step B (50 mg, 0.16 mmol), 4-trifluoromethylbenzoyl chloride (0.16 mL of 1N solution in THF, 0.16 mmol) and triethylamine (0.16 mL of 2N solution in CH₂Cl₂, 0.32 nmol) were dissolved in anhydrous THF (2 ml). The reaction mixture was stirred at room temperature under N₂ for 1 hour. The solvent was evaporated. The resulting residue was partitioned between water and EtOAc. The organic layer was dried over MgSO₄, filtered and concentrated. The crude product was subject to MPLC to give the title compound as white powder (47 mg, 0.11 mmol, 65% yield). MS 464.1 (M+Na)⁺.

### Step D: 5-Biphenyl-4-yl-2-(4-trifluoromethylphenyl)-1H-imidazole-4-carboxylic acid methyl ester

3-Biphenyl-4-yl-3-oxo-2-(4-trifluoromethylbenzoylamino)propionic acid methyl ester from Step D (80 mg, 0.181 mmol), ammonium acetate (70 mg, 0.91 mmol) and acetic acid (0.1 ml) were added to o-xylene (3 ml). The mixture was refluxed for 15 hours. The reaction mixture was concentrated and partitioned between EtOAc and NaHCO₃ solution (3 N). The organic layer was dried over MgSO₄, filtered and concentrated. The resulting residue was subject to MPLC to give the title compound (60 mg, 0.142 mmol, 79%). MS 423.1 (M+H)⁺.

### Step E: 5-Biphenyl-4-yl-2-(4-trifluoromethyl-phenyl)-1H-imidazole-4-carboxylic acid

5-Biphenyl-4-yl-2-(4-trifluoromethylphenyl)-1H-imidazole-4-carboxylic acid methyl ester from Step D (60 mg, 0.142 mmol) was dissolved in methanol (3 ml) and NaOH solution (1 N, 0.5 ml) was added. The reaction mixture was refluxed for 15 hours. The solvent was evaporated. The resulting water solution was partitioned between HCl solution (1 N) and EtOAc. The water layer was extracted with EtOAc. The organic layers were combined, dried over MgSO₄, and concentrated to give the title compound as a white solid, which was used without further purification. MS 409.0 (M+H)⁺.

### Step F: 3-Benzylsulfanyl-2-{[5-biphenyl-4-yl-2-(4-trifluoromethylphenyl)-1H-imidazole-4-carbonyl]amino}propionic acid methyl ester

5-Biphenyl-4-yl-2-(4-trifluoromethylphenyl)-1H-imidazole-4-carboxylic acid from Step E (68 mg, crude, 0.17 mmol), (L)-S-benzylcysteine methyl ester hydrochloride (45 mg, 0.17 mmol), PyBop (87 mg, 0.17 mmol) and triethylamine (1N, 0.34 ml, 0.34 mmol) were dissolved in CH₂Cl₂ (3 ml). The mixture was stirred at room temperature for 3 hours. The mixture was concentrated, and subject to MPLC to give the title compound (12 mg, 0.0194 mmol, 14% yield over Steps E-F). MS 614.0 (M−H)⁻.

### Step G: (R)-2-{[5-Biphenyl-4-yl-2-(4-trifluoromethylphenyl)-1H-imidazole-4-carbonyl]amino}-3-phenylmethanesulfinylpropionic acid

3-Benzylsulfanyl-2-{[5-biphenyl-4-yl-2-(4-trifluoromethylphenyl)-1H-imidazole-4-carbonyl]amino}propionic acid methyl ester from Step F (12 mg, 0.0194 mmol) was dissolved in THF (2 ml) and NaOH solution (1 N, 1 ml) was added. The reaction mixture was stirred at room temperature for 5 hours. The THF was evaporated. The aqueous solution was acidified to pH 1 with HCl (1 N). The mixture was concentrated followed by HPLC purification of the residue giving 5 mg of the title compound as diastereomers. MS 618.0 (M+H)⁺.

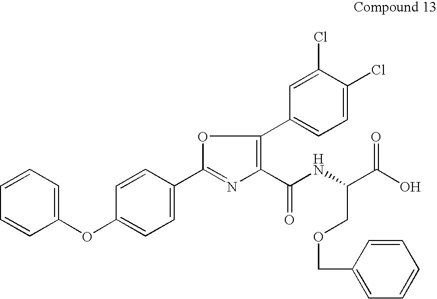

### (S)-3-Benzyloxy-2-{[5-(3,4-dichlorophenyl)-2-(4-phenoxyphenyl)-oxazole-4-carbonyl]amino}propionic acid

The title compound was prepared by a procedure analogous to that of Compound 1 by substituting 3,4-dichlorobenzoyl chloride for the 4-cyanobenzoyl chloride of Step A of Compound 1; and by substituting 1-iodo-4-phenoxybenzene for the 1-iodo-4-trifluoromethylbenzene of Step B of Compound 1; and by substituting H-Ser(Bzl)-L-OMe hydrochloride for the H-Cys(Bzl)-L-OMe hydrochloride of Step D of Compound 1. MS 601.0 (M−H)⁻.

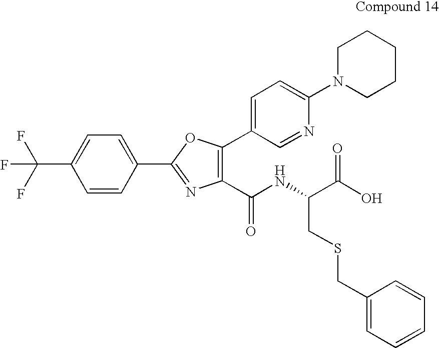

### (R)-3-Benzylsulfanyl-2-{[5-(3,4,5,6-tetrahydro-2H-[1,2′]bipyridin-5′-yl)-2-(4-trifluoromethylphenyl)oxazole-4-carbonyl]amino}propionic acid

### Step A: 5-(6-Chloropyridin-3-yl)oxazole-4-carboxylic acid methyl ester

The title compound was prepared by a procedure analogous to that of Step A of Compound 1 by substituting 6-chloronicotinoyl chloride for the 4-cyanobenzoyl chloride of Step A of Compound 1. MS 239.0 (M+H)⁺.

### Step B: 5-(3,4,5,6-Tetrahydro-2H-[1,2′]bipyridin-5′-yl)oxazole-4-carboxylic acid methyl ester

To a solution of 5-(6-chloropyridin-3-yl)oxazole-4-carboxylic acid methyl ester from Step A (100 mg, 0.42 mmol) in dichloromethane (1.0 mL) at 0° C. was added piperidine (0.08 mL, 0.84 mmol) and the resulting reaction mixture was stirred at 0° C. for 30 min and then at room temperature for an additional 12 h. More dichloromethane (2.0 mL) was added to the mixture and it was washed with water (3 mL×3). The organic layer was separated, dried over sodium sulfate and the filtrate was concentrated in vacuo to give 120 mg (100%) of the title compound, which was used without further purification. MS 288.0 (M+H)⁺.

### Step C: 5-(3,4,5,6-Tetrahydro-2H-[1,2′]bipyridin-5′-yl)-2-(4-trifluoromethylphenyl) oxazole-4-carboxylic acid methyl ester

The title compound was prepared by a procedure analogous to that of Step B of Compound 1 by substituting 5-(3,4,5,6-tetrahydro-2H-[1,2′]bipyridin-5′-yl)oxazole-4-carboxylic acid methyl ester for the 5-(4-cyanophenyl)oxazole-4-carboxylic acid methyl ester of Step B of Compound 1. MS 432.0 (M+H)⁺.

### Step D: (R)-3-Benzylsulfanyl-2-{[5-(3,4,5,6-tetrahydro-2H-[1,2′]bipyridin-5′-yl)-2-(4-trifluoromethylphenyl)-oxazole-4-carbonyl]amino}propionic acid

The title compound was prepared by a procedure analogous to that of Steps C-E of Compound 1 by substituting 5-(3,4,5,6-Tetrahydro-2H-[1,2′]bipyridin-5′-yl)-2-(4-trifluoromethylphenyl)-oxazole-4-carboxylic acid methyl ester for the 5-(4-cyanophenyl)-2-(4-trifluoromethylphenyl)oxazole-4-carboxylic acid methyl ester of Step C of Compound 1. MS 611.0 (M+H)⁺.

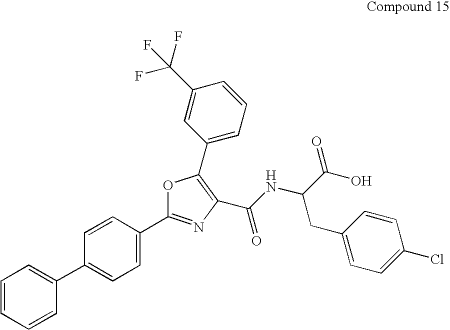

### 2-{[2-Biphenyl-4-yl-5-(3-trifluoromethylphenyl)oxazole-4-carbonyl]amino}-3-(4-chlorophenyl)propionic acid

The title compound was prepared by a procedure analogous to that of Compound 1 by substituting 3-trifluoromethylbenzoyl chloride for the 4-cyanobenzoyl chloride of Step A of Compound 1; and by substituting 4-bromobiphenyl for the 1-iodo-4-trifluoromethylbenzene of Step B of Compound 1; and by substituting D,L-4-chlorophenylalanine ethyl ester hydrochloride for the H-Cys(Bzl)-L-OMe hydrochloride of Step D of Compound 1. MS 589.0 (M−H)⁻.

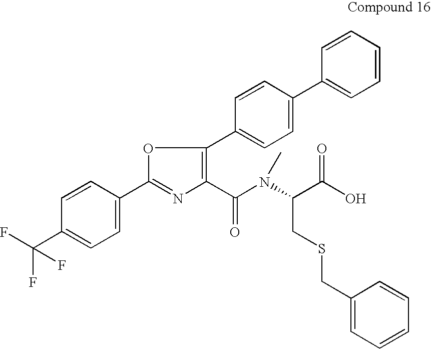

### (R)-3-Benzylsulfanyl-2-{[5-biphenyl-4-yl-2-(4-trifluoromethylphenyl)oxazole-4-carbonyl]methylamino}propionic acid

(R)-3-Benzylsulfanyl-2-{[5-biphenyl-4-yl-2-(4-trifluoromethylphenyl)-oxazole-4-carbonyl]amino}propionic acid methyl ester from Step D of Compound 1 (90 mg, 0.146 mmol) was dissolved in THF (anhydrous, 2 ml). The solution was cooled to 0° C. Sodium hydride (4.2 mg, 0.175 mmol) was added under N₂. After 30 min, methyl iodide (0.01 ml, 0.175 mmol) was added to the reaction mixture. The reaction was allowed to warm to room temperature slowly. After 16 hours, the reaction mixture was concentrated in vacuo, diluted with water (2 ml) and EtOAc (10 ml), and acidified with 1N HCl to pH 3. The organic layer was then separated, dried over MgSO₄, filtered and the filtrate was concentrated in vacuo. The crude product was subject to reverse phase HPLC to give the title compound as a white solid (9 mg, 0.146 mmol, 10% yield). MS 617.1 (M+H)⁺.

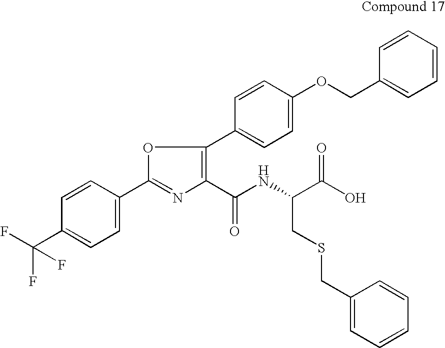

### (R)-2-{[5-(4-Benzyloxyphenyl)-2-(4-trifluoromethylphenyl)oxazole-4-carbony]amino}-3-benzylsulfanylpropionic acid

The title compound was prepared by a procedure analogous to that of Compound 1 by substituting 4-benzyloxybenzoyl chloride for the 4-cyanobenzoyl chloride of Step A of Compound 1. MS 631.0 (M−H)⁻.

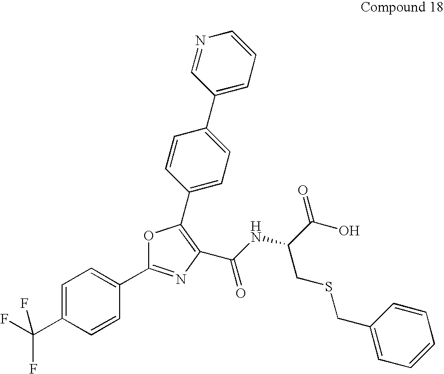

### (R)-3-Benzylsulfanyl-2-{[5-(4-pyridin-3-yl-phenyl)-2-(4-trifluoromethylphenyl)-oxazole-4-carbonyl]amino}propionic acid

### Step A: 5-(4-Acetoxyphenyl)-2-(4-trifluoromethylphenyl)oxazole-4-carboxylic acid ethyl ester

The title compound was prepared by a procedure analogous to that of Steps A-B of Compound 1 by substituting acetic acid 4-chlorocarbonylphenyl ester for the 4-cyanobenzoyl chloride, and ethyl isocyanoacetate for the methyl isocyanoacetate of Step A of Compound 1.

### Step B: 5-(4-Hydroxyphenyl)-2-(4-trifluoromethylphenyl)oxazole-4-carboxylic acid ethyl ester

5-(4-Acetoxyphenyl)-2-(4-trifluoromethylphenyl)oxazole-4-carboxylic acid ethyl ester from Step A (2.66 g, 6.35 mmol) was dissolved in THF (50 ml) followed by addition of 6.35 ml 3 N NaOH solution. The reaction was stirred at room temperature for 16 hours. The reaction mixture was concentrated on a rotovap. The resulting aqueous solution was acidified with 1N HCl to pH 2. A white precipitate formed and was filtered, washed with H₂O (20 ml), then washed with hexane (5 ml) to give the title compound as a white solid (2.39 g, 6.4 mmol, 100% yield). MS 378.0 (M+H)⁺.

### Step C: 5-(4-Trifluoromethanesulfonyloxyphenyl)-2-(4-trifluoromethylphenyl)oxazole-4-carboxylic acid ethyl ester

A mixture of 5-(4-Hydroxyphenyl)-2-(4-trifluoromethylphenyl)oxazole-4-carboxylic acid ethyl ester from Step B (0.5 g, 1.33 mmol), triflic anhydride (0.449 g, 1.59 mmol) and triethylamine (0.322 g, 3.18 mmol) in dichloromethane (anhydrous, 5 ml) at 0° C. under N₂ was allowed to warm to room temperature and was stirred for 3 h. The reaction mixture was concentrated in vacuo, and diluted with water and EtOAc. After adjusting the pH to ˜7, the organic layer was separated, dried over MgSO₄, filtered, and the filtrate was concentrated in vacuo. Purification by MPLC on silica gel gave the title compound. MS 510.0 (M+H)⁺.

### Step D: 5-[4-(4,4,5,5-Tetramethyl[1,3,2]dioxaborolan-2-yl)phenyl]-2-(4-trifluoromethyl phenyl)oxazole-4-carboxylic acid ethyl ester

A mixture of 5-(4-Trifluoromethanesulfonyloxyphenyl)-2-(4-trifluoromethyl-phenyl)oxazole-4-carboxylic acid ethyl ester from Step C (0.36 g, 0.707 mmol), bis(pinacolato)diboron (0.20 g, 0.778 mmol), PdCl₂(dppf) (15.5 mg, 0.02 mmol), dppf (12 mg, 0.02 mmol) and potassium acetate (0.208 g, 2.121 mmol) in 3 ml of dioxane was heated at 80° C. for 16 h. Another 0.2 g of bis(pinacolato)diboron was added and the reaction mixture was stirred for an additional 3 h. The mixture was cooled to room temperature and diluted with EtOAc and water. The organic layer was separated, washed with brine, dried over MgSO₄, filtered, and the filtrate was concentrated in vacuo. Purification by MPLC on silica gel gave the title compound (0.42 g, 83% pure). MS 488.1 (M+H)⁺.

### Step E: 5-(4-Pyridin-3-ylphenyl)-2-(4-trifluoromethylphenyl)oxazole-4-carboxylic acid ethyl ester

A mixture of 5-[4-(4,4,5,5-Tetramethyl[1,3,2]dioxaborolan-2-yl)phenyl]-2-(4-trifluoromethylphenyl)oxazole-4-carboxylic acid ethyl ester from Step D (0.2 mmol), PdCl₂(dppf) (10.2 mg, 0.014 mmol), potassium carbonate (55 mg, 0.4 mmol) and 3-bromopyridine (0.023 ml, 0.24 mmol) in dioxane (2 ml) was stirred at 80° C. for 16 h. The mixture was cooled to room temperature and diluted with EtOAc and water. The organic layer was separated, washed with brine, dried over MgSO₄, filtered, and the filtrate was concentrated in vacuo. Purification by MPLC on silica gel gave the title compound.

### Step F: 5-(4-Pyridin-3-yl-phenyl)-2-(4-trifluoromethylphenyl)oxazole-4-carboxylic acid

5-(4-Pyridin-3-yl-phenyl)-2-(4-trifluoromethylphenyl)oxazole-4-carboxylic acid ethyl ester from Step E was dissolved in THF (3 ml) and NaOH (1 N, 1 ml) was added. The reaction mixture was heated at reflux temperature for 16 h. The mixture was concentrated in vacuo and acidified to pH 2 with 1N HCl. A white precipitate formed, and was filtered to give the title compound (45 mg, 0.11 mmol, 55% yield over Steps E-F). MS 411.0 (M+H)⁺.

### Step G: (R)-3-Benzylsulfanyl-2-{[5-(4-pyridin-3-yl-phenyl)-2-(4-trifluoromethylphenyl) oxazole-4-carbonyl]amino}propionic acid

The title compound was prepared by a procedure analogous to that of Steps D-E of Compound 1 by substituting 5-(4-pyridin-3-yl-phenyl)-2-(4-trifluoromethylphenyl)oxazole-4-carboxylic acid from Step F of Compound 18 for the 5-(4-cyanophenyl)-2-(4-trifluoromethylphenyl)oxazole-4-carboxylic acid of Step D of Compound 1. MS 604.0 (M+H)⁺.

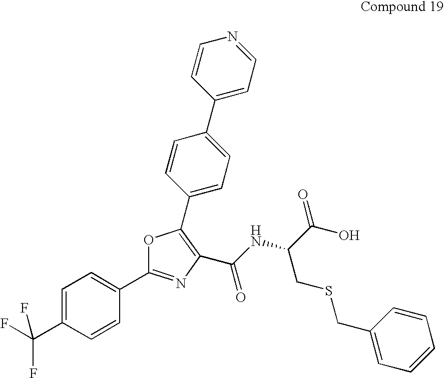

### (R)-3-Benzylsulfanyl-2-{[5-(4-pyridin-4-yl-phenyl)-2-(4-trifluoromethylphenyl)oxazole-4-carbonyl]amino}propionic acid

The title compound was prepared by a procedure analogous to that of Compound 18 by substituting 4-bromopyridine for the 3-bromopyridine of Step E of Compound 18. MS 604.0 (M+H)⁺.

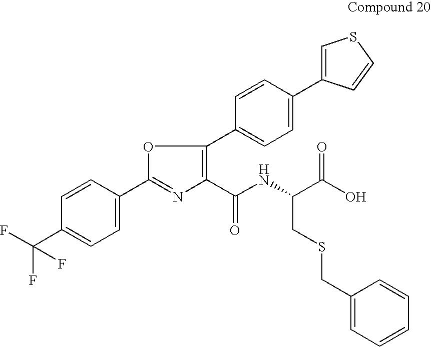

### (R)-3-Benzylsulfanyl-2-{[5-(4-thien-3-yl-phenyl)-2-(4-trifluoromethylphenyl)oxazole-4-carbonyl]amino}propionic acid

The title compound was prepared by a procedure analogous to that of Compound 18 by substituting 3-bromothiophene for the 3-bromopyridine of Step E of Compound 18. MS 609.0 (M+H)⁺.

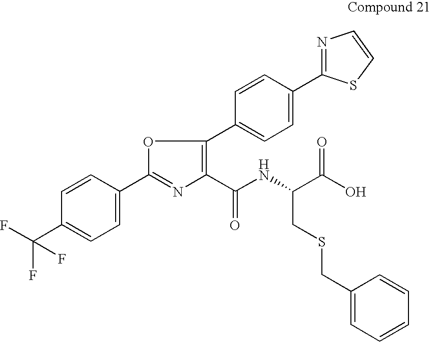

### (R)-3-Benzylsulfanyl-2-{[5-(4-thiazol-2-yl-phenyl)-2-(4-trifluoromethylphenyl)oxazole-4-carbonyl]amino}propionic acid

The title compound was prepared by a procedure analogous to that of Compound 18 by substituting 2-bromothiazole for the 3-bromopyridine of Step E of Compound 18. MS 610.0 (M+H)⁺.

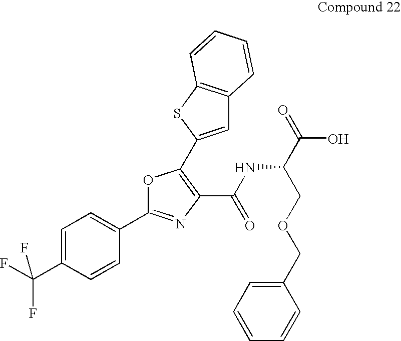

### (S)-2-{[5-Benzo[b]thien-2-yl-2-(4-trifluoromethylphenyl)oxazole-4-carbonyl]amino}-3-benzyloxypropionic acid

The title compound was prepared by a procedure analogous to that of Compound 1 by substituting benzo[b]thiophene-2-carbonyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 566.0 (M−H)⁻.

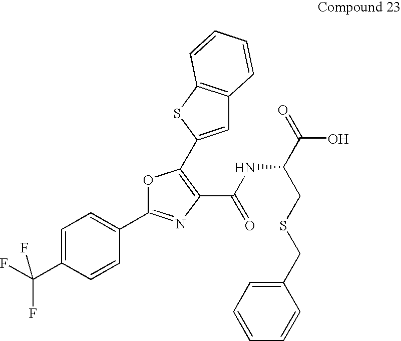

### (R)-2-{[5-Benzo[b]thien-2-yl-2-(4-trifluoromethylphenyl)oxazole-4-carbonyl]amino}-3-benzylsulfanylpropionic acid

The title compound was prepared by a procedure analogous to that of Compound 1 by substituting benzo[b]thiophene-2-carbonyl chloride for the 4-cyanobenzoyl chloride of Step A of Compound 1. MS 581.0 (M−H)⁻.

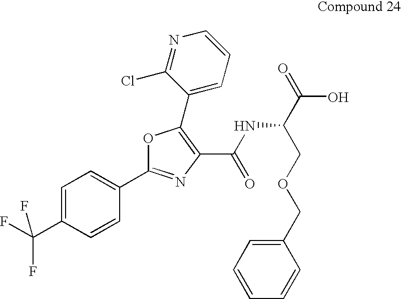

### (S)-3-Benzyloxy-2-{[5-(2-chloropyridin-3-yl)-2-(4-trifluoromethylphenyl)oxazole-4-carbonyl]amino}propionic acid

The title compound was prepared by a procedure analogous to that of Compound 1 by substituting 2-chloronicotinoyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 544.0 (M−H)⁻.

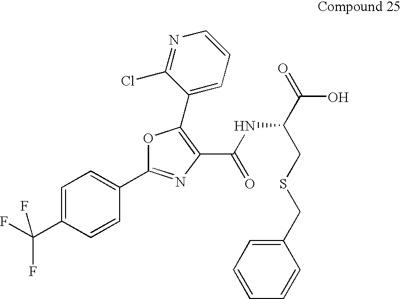

### (R)-3-Benzylsulfanyl-2-{[5-(2-chloropyridin-3-yl)-2-(4-trifluoromethylphenyl)oxazole-4-carbonyl]amino}propionic acid

The title compound was prepared by a procedure analogous to that of Compound 1 by substituting 2-chloronicotinoyl chloride for the 4-cyanobenzoyl chloride of Step A of Compound 1. MS 560.1 (M−H)⁻.

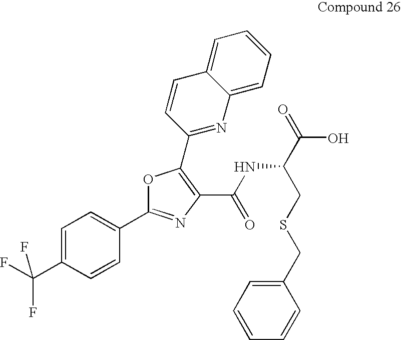

### (R)-3-Benzylsulfanyl-2-{[5-quinolin-2-yl-2-(4-trifluoromethylphenyl)oxazole-4-carbonyl]amino}propionic acid

### Step A: Quinoline-2-carbonyl chloride

Quinoline-2-carboxylic acid (5 g, 28.9 mmol) was suspended in thionyl chloride (6.32 ml, 86.6 mmol). The reaction mixture was heated at 60° C. for 6 h. The heterogeneous mixture became a homogeneous solution. The solution was concentrated in vacuo to give the title compound as a yellow powder (5.6 g, 29 mmol, 100% yield). MS 255.1 (M+H)⁺.

### Step B: (R)-3-Benzylsulfanyl-2-{[5-guinolin-2-yl-2-(4-trifluoromethylphenyl)oxazole-4-carbonyl]amino}propionic acid

The title compound was prepared by a procedure analogous to that of Compound 1 by substituting quinoline-2-carbonyl chloride for the 4-cyanobenzoyl chloride of Step A of Compound 1. MS 576.1 (M−H)⁻.

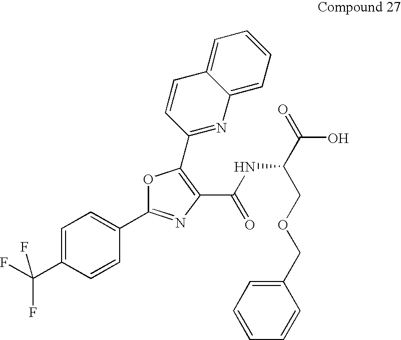

### (S)-3-Benzyloxy-2-{[5-quinolin-2-yl-2-(4-trifluoromethylphenyl)oxazole-4-carbonyl]amino}propionic acid

The title compound was prepared by a procedure analogous to that of Compound 26 by substituting H-Ser(Bzl)-L-OMe hydrochloride for the H-Cys(Bzl)-L-OMe hydrochloride of Step B of Compound 26. MS 560.1 (M−H)⁻.

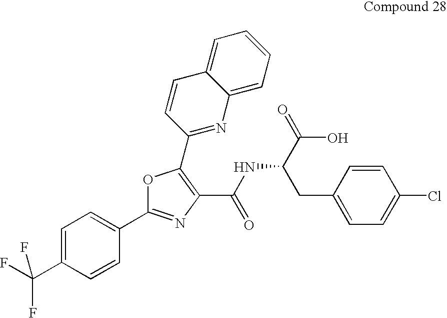

### (S)-3-(4-Chlorophenyl)-2-{[5-quinolin-2-yl-2-(4-trifluoromethylphenyl)oxazole-4-carbonyl]amino}propionic acid

The title compound was prepared by a procedure analogous to that of Compound 26 by substituting L-4-chlorophenylalanine ethyl ester hydrochloride for the H-Cys(Bzl)-L-OMe hydrochloride of Step B of Compound 26. MS 564.0 (M−H)⁻.

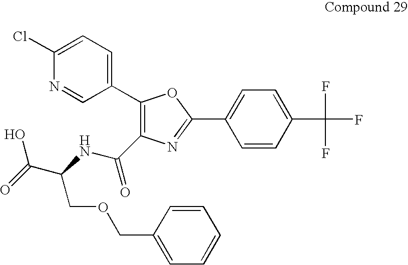

### (S)-3-Benzyloxy-2-{[5-(6-chloropyridin-3-yl)-2-(4-trifluoromethylphenyl)oxazole-4-carbonyl]amino}propionic acid

The title compound was prepared by a procedure analogous to that of Compound 1 by substituting 6-chloronicotinoyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 544.1 (M−H)⁻.

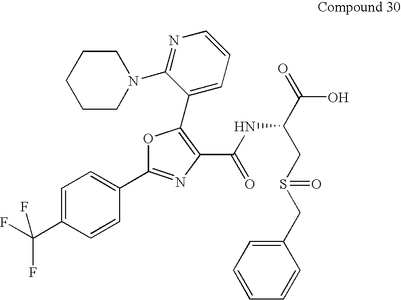

### (R)-3-Phenylmethanesulfinyl-2-{[5-(3,4,5,6-tetrahydro-2H-[1,2′]bipyridin-3′-yl)-2-(4-trifluoromethylphenyl)oxazole-4-carbonyl]amino}propionic acid

### Step A: 5-(2-Chloropyridin-3-yl)-oxazole-4-carboxylic acid tert-butyl ester.

A mixture of 2-chloronicotinoyl chloride (3 g, 17 mmol), t-butyl isocyanoacetate (2.5 ml, 17 mmol) and triethylamine (4.54 ml, 34 mmol) in THF (25 mL) was stirred at room temperature for 3 h. It was then heated at 50° C. for 10 h. The mixture was cooled to room temperature and filtered through Celite. The filtrate was concentrated in vacuo and purified by medium pressure liquid chromatography on silica gel to give the title compound as a light brown solid (0.76 g, 2.7 mmol, 16% yield). MS 281.1 (M+H)⁺.

### Step B: 5-(2-Chloro-pyridin-3-yl)-2-(4-trifluoromethylphenyl)oxazole-4-carboxylic acid tert-butyl ester

The title compound was prepared by a procedure analogous to that of Step B of Compound 1 by substituting 5-(2-chloropyridin-3-yl)oxazole-4-carboxylic acid tert-butyl ester of Step A of Compound 30 for the 5-(4-cyanophenyl)oxazole-4-carboxylic acid methyl ester of Step B of Compound 1. MS 871.0 (2M+Na)⁺.

### Step C: 5-(3,4,5,6-Tetrahydro-2H-[1,2′]bipyridin-3′-yl)-2-(4-trifluoromethylphenyl)-oxazole-4-carboxylic acid tert-butyl ester

A mixture of 5-(2-chloropyridin-3-yl)-2-(4-trifluoromethylphenyl)oxazole-4-carboxylic acid tert-butyl ester from Step B (81 mg, 0.191 mmol) and piperidine (1.2 ml, 1.15 mmol) was heated at 80° C. for 15 h. The reaction mixture was concentrated in vacuo and diluted with EtOAc and water. The organic layer was collected, dried over MgSO₄ and concentrated in vacuo. Purification by medium pressure liquid chromatography on silica gel gave the title compound as a white solid. MS 474.1 (M+H)⁺.

### Step D: 5-(3,4,5,6-Tetrahydro-2H-[1,2′]bi din-3′-yl)-2-(4-trifluoromethylphenl)-oxazole-4-carboxylic acid

A mixture of 5-(3,4,5,6-tetrahydro-2H-[1,2′]bipyridin-3′-yl)-2-(4-trifluoromethylphenyl)oxazole-4-carboxylic acid tert-butyl ester from Step C in CH₂Cl₂ (4 ml) and TFA (2 ml) was stirred at room temperature for 0.6 h. The solvent was evaporated and the crude oil was dissolved in CH₂Cl₂ (20 ml). The solution was concentrated in vacuo and dried to give the title compound as a light yellow oil (48 mg, 0.115 mmol, 60% yield over Steps C-D).

### Step E: (R)-3-Phenylmethanesulfinyl-2-{[5-(3,4,5,6-tetrahydro-2H-[1,2′]bipyridin-3′-yl)-2-(4-trifluoromethylphenyl)oxazole-4-carbonyl]amino}propionic acid

The title compound was prepared by a procedure analogous to that of Steps D-E of Compound 1 by substituting 5-(3,4,5,6-tetrahydro-2H-[1,2′]bipyridin-3′-yl)-2-(4-trifluoromethylphenyl)oxazole-4-carboxylic acid of Step D of Compound 30 for the 5-(4-cyanophenyl)-2-(4-trifluoromethylphenyl)oxazole-4-carboxylic acid of Step D of Compound 1. MS 627.2 (M+H)⁺.

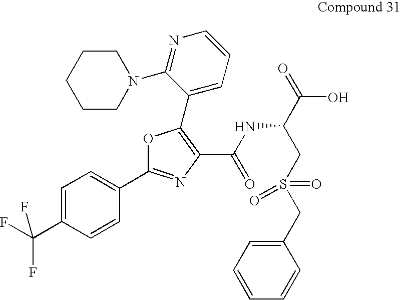

### (R)-3-Phenylmethanesulfonyl-2-{[5-(3,4,5,6-tetrahydro-2H-[1,2′]bipyridin-3′-yl)-2-(4-trifluoromethylphenyl)oxazole-4-carbonyl]amino}propionic acid

The title compound was a by-product from Step E of Compound 30. MS 641.1 (M−H)⁻.

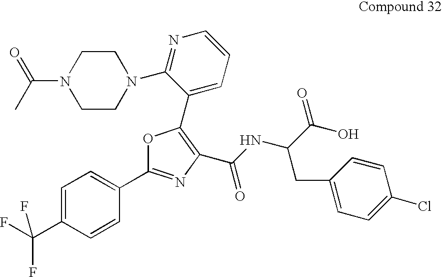

### 2-{[5-[2-(4-Acetylpiperazin-1-yl)pyridin-3-yl]-2-(4-trifluoromethylphenyl)oxazole-4-carbonyl]amino}-3-(4-chlorophenyl)propionic acid

The title compound was prepared by a procedure analogous to that of Compound by substituting 1-piperazin-1-yl-ethanone for the piperidine of Step C of Compound 30. MS 640.1 (M−H)⁻.

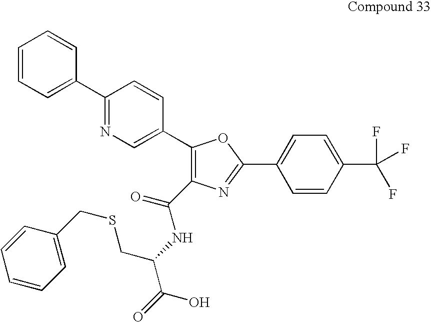

### (R)-3-Benzylsulfanyl-2-{[5-(6-phenylpyridin-3-yl)-2-(4-trifluoromethylphenyl)oxazole-4-carbonyl]amino}propionic acid

### Step A: 5-(6-Chloropyridin-3-yl)-2-(4-trifluoromethylphenyl)oxazole-4-carboxylic acid methyl ester

The title compound was prepared by a procedure analogous to that of Steps A-B of Compound 1 by substituting 6-chloronicotinoyl chloride for the 4-cyanobenzoyl chloride of Step A of Compound 1. MS 384.1 (M+H)⁺.

### Step B: 5-(6-Phenylpyridin-3-yl)-2-(4-trifluoromethylphenyl)oxazole-4-carboxylic acid methyl ester

A mixture of 5-(6-chloropyridin-3-yl)-2-(4-trifluoromethylphenyl)oxazole-4-carboxylic acid methyl ester from Step A (191 mg, 0.5 mmol), phenylboronic acid (61 mg, 0.5 mmol), cesium fluoride (152 mg, 1.0 mmol) and palladium bistri-tert-butylphosphine (13 mg, 5% mmol) in DMF (2.0 mL) was heated at reflux temperature for 14 h. The reaction mixture was then cooled to room temperature and filtered through a pad of Celite. The filtrate was concentrated in vacuo. Purification by medium pressure liquid chromatography on silica gel (1:9 ethyl acetate/hexanes) gave 148 mg (70%) of the title compound. MS 425.1 (M+H)⁺.

### Step C: (R)-3-Benzylsulfanyl-2-{[5-(6-phenylpyridin-3-yl-2-(4-trifluoromethylphenyl) oxazole-4-carbonyl]amino}propionic acid

The title compound was prepared by a procedure analogous to that of Steps C-E of Compound 1 by substituting 5-(6-phenylpyridin-3-yl)-2-(4-trifluoromethylphenyl)oxazole-4-carboxylic acid methyl ester for the 5-(4-cyanophenyl)-2-(4-trifluoromethylphenyl)oxazole-4-carboxylic acid methyl ester of Step C of Compound 1. MS 605.1 (M+H)⁺.

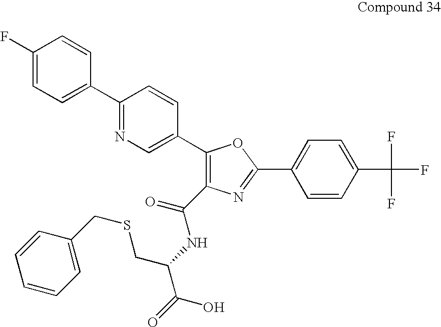

### (R)-3-Benzylsulfanyl-2-{[5-[6-(4-fluorophenyl)pyridin-3-yl]-2-(4-trifluoromethylphenyl)oxazole-4-carbonyl]amino}propionic acid

The title compound was prepared by a procedure analogous to that of Compound 33 by substituting 4-fluorophenylboronic acid for the phenylboronic acid of Step B of Compound 33. MS 622.0 (M+H)⁺.

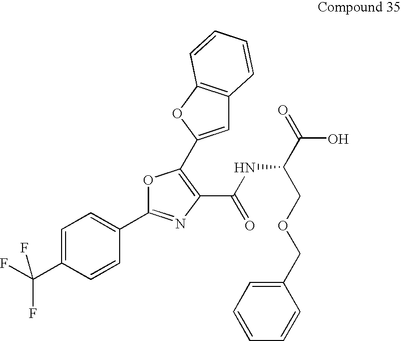

### (S)-2-{[5-Benzofuran-2-yl-2-(4-trifluoromethylphenyl)oxazole-4-carbonyl]amino}-3-benzyloxypropionic acid

The title compound was prepared by a procedure analogous to that of Compound 1 by substituting benzo[b]furan-2-carbonyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 549.2 (M−H)⁻.

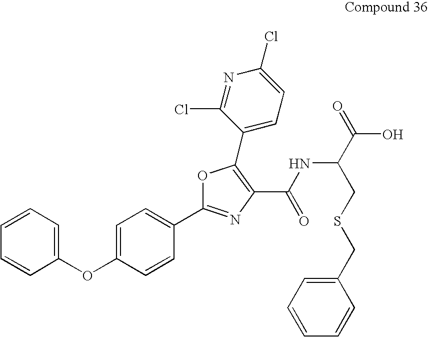

### 3-Benzylsulfanyl-2-{[5-(2,6-dichloropyridin-3-yl)-2-(4-phenoxyphenyl)oxazole-4-carbonyl]amino}propionic acid

The title compound was prepared by a procedure analogous to that of Compound 1 by substituting 2,6-dichloronicotinoyl chloride for the 4-cyanobenzoyl chloride of Step A of Compound 1; and by substituting 1-iodo-4-phenoxybenzene for the 1-iodo-4-trifluoromethylbenzene of Step B of Compound 1. MS 618.0 (M−H)⁻.

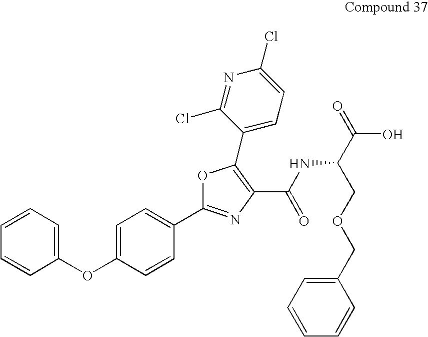

### (S)-3-Benzyloxy-2-{[5-(2,6-dichloropyridin-3-yl)-2-(4-phenoxyphenyl)oxazole-4-carbonyl]amino}propionic acid

The title compound was prepared by a procedure analogous to that of Compound 1 by substituting 2,6-dichloronicotinoyl chloride for the 4-cyanobenzoyl chloride of Step A of Compound 1; and by substituting 1-iodo-4-phenoxybenzene for the 1-iodo-4-trifluoromethylbenzene of Step B of Compound 1; and by substituting H-Ser(Bzl)-L-OMe hydrochloride for the H-Cys(Bzl)-L-OMe hydrochloride of Step D of Compound 1. MS 603.9 (M−H)⁻.

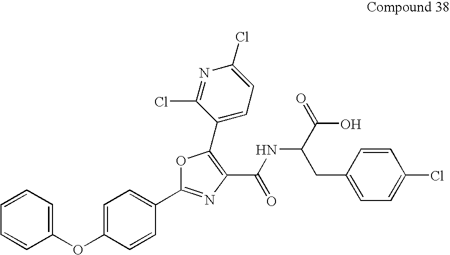

### 3-(4-Chlorophenyl)-2-{[5-(2,6-dichloropyridin-3-yl)-2-(4-phenoxyphenyl)oxazole-4-carbonyl]amino}propionic acid

The title compound was prepared by a procedure analogous to that of Compound 1 by substituting 2,6-dichloronicotinoyl chloride for the 4-cyanobenzoyl chloride of Step A of Compound 1; and by substituting 1-iodo-4-phenoxybenzene for the 1-iodo-4-trifluoromethylbenzene of Step B of Compound 1; and by substituting D,L-4-chlorophenylalanine ethyl ester hydrochloride for the H-Cys(Bzl)-L-OMe hydrochloride of Step D of Compound 1. MS 608.0 (M+H)⁺.

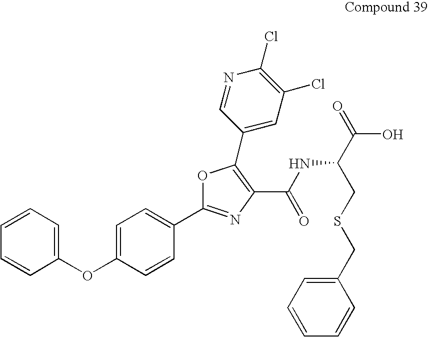

### (R)-3-Benzylsulfanyl-2-{[5-(5,6-dichloropyridin-3-yl)-2-(4-phenoxyphenyl)oxazole-4-carbonyl]amino}propionic acid

The title compound was prepared by a procedure analogous to that of Compound 1 by substituting 5,6-dichloronicotinoyl chloride for the 4-cyanobenzoyl chloride of Step A of Compound 1; and by substituting 1-iodo-4-phenoxybenzene for the 1-iodo-4-trifluoromethylbenzene of Step B of Compound 1. MS 618.0 (M−H)⁻.

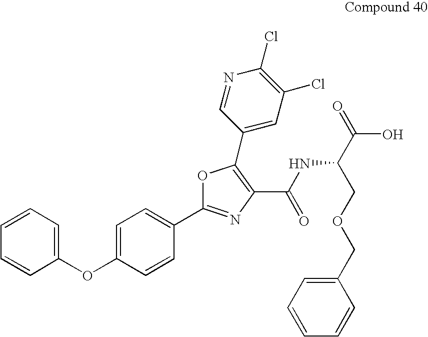

### (S)-3-Benzyloxy-2-{[5-(5,6-dichloropyridin-3-yl)-2-(4-phenoxyphenyl)oxazole-4-carbonyl]amino}propionic acid

The title compound was prepared by a procedure analogous to that of Compound 1 by substituting 5,6-dichloronicotinoyl chloride for the 4-cyanobenzoyl chloride of Step A of Compound 1; and by substituting 1-iodo-4-phenoxybenzene for the 1-iodo-4-trifluoromethylbenzene of Step B of Compound 1; and by substituting H-Ser(Bzl)-L-OMe hydrochloride for the H-Cys(Bzl)-L-OMe hydrochloride of Step D of Compound 1. MS 604.0 (M+H)⁺.

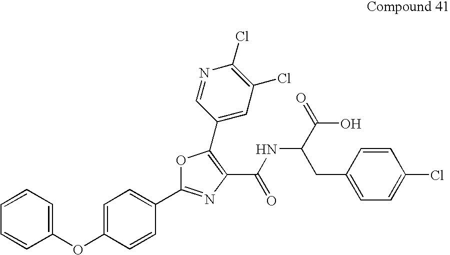

### 3-(4-Chlorophenyl)-2-{[5-(5,6-dichloropyridin-3-yl)-2-(4-phenoxyphenyl)oxazole-4-carbonyl]amino}propionic acid

The title compound was prepared by a procedure analogous to that of Compound 1 by substituting 5,6-dichloronicotinoyl chloride for the 4-cyanobenzoyl chloride of Step A of Compound 1; and by substituting 1-iodo-4-phenoxybenzene for the 1-iodo-4-trifluoromethylbenzene of Step B of Compound 1; and by substituting D,L-4-chlorophenylalanine ethyl ester hydrochloride for the H-Cys(Bzl)-L-OMe hydrochloride of Step D of Compound 1. MS 608.0 (M+H)⁺.

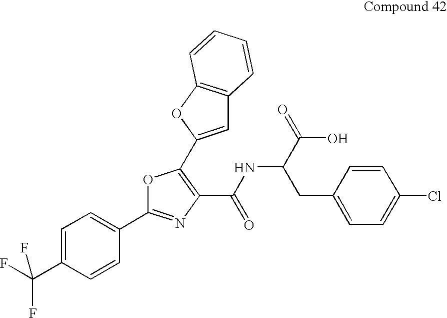

### 2-{[5-Benzofuran-2-yl-2-(4-trifluoromethylphenyl)oxazole-4-carbonyl]amino}-3-(4-chlorophenyl)propionic acid

The title compound was prepared by a procedure analogous to that of Compound 1 by substituting benzo[b]furan-2-carbonyl chloride for the 4-cyanobenzoyl chloride of Step A of Compound 1; and by substituting D,L-4-chlorophenylalanine ethyl ester hydrochloride for the H-Cys(Bzl)-L-OMe hydrochloride of Step D of Compound 1. MS 555.0 (M+H)⁺.

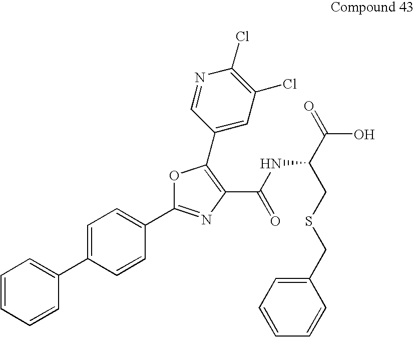

### (R)-3-Benzylsulfanyl-2-{[2-biphenyl-4-yl-5-(5,6-dichloropyridin-3-yl)oxazole-4-carbonyl]amino}propionic acid

The title compound was prepared by a procedure analogous to that of Compound 1 by substituting 5,6-dichloronicotinoyl chloride for the 4-cyanobenzoyl chloride of Step A of Compound 1; and by substituting 4-bromobiphenyl for the 1-iodo-4-trifluoromethylbenzene of Step B of Compound 1. MS 602.1 (M−H)⁻.

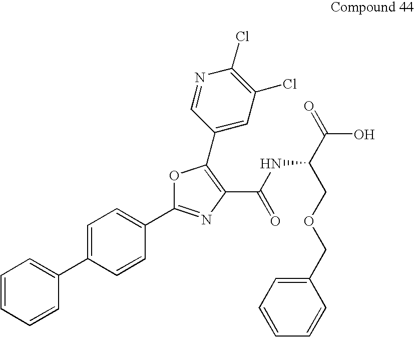

### (S)-3-Benzyloxy-2-{[2-biphenyl-4-yl-5-(5,6-dichloropyridin-3-yl)oxazole-4-carbonyl]amino}propionic acid

The title compound was prepared by a procedure analogous to that of Compound 1 by substituting 5,6-dichloronicotinoyl chloride for the 4-cyanobenzoyl chloride of Step A of Compound 1; and by substituting 4-bromobiphenyl for the 1-iodo-4trifluoromethylbenzene of Step B of Compound 1; and by substituting H-Ser(Bzl)-L-OMe hydrochloride for the H-Cys(Bzl)-L-OMe hydrochloride of Step D of Compound 1. MS 588.0 (M+H)⁺.

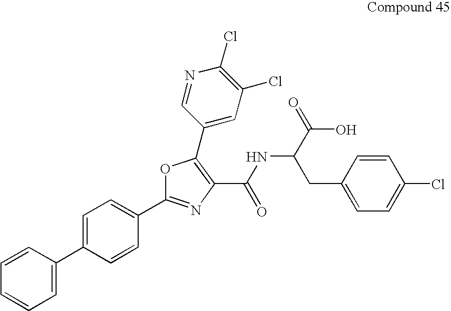

### 2-{[2-Biphenyl-4-yl-5-(5,6-dichloropyridin-3-yl)oxazole-4-carbonyl]amino}-3-(4-chlorophenyl)propionic acid

The title compound was prepared by a procedure analogous to that of Compound 1 by substituting 5,6-dichloronicotinoyl chloride for the 4-cyanobenzoyl chloride of Step A of Compound 1; and by substituting 4-bromobiphenyl for the 1-iodo-4-trifluoromethylbenzene of Step B of Compound 1; and by substituting D,L-4-chlorophenylalanine ethyl ester hydrochloride for the H-Cys(Bzl)-L-OMe hydrochloride of Step D of Compound 1. MS 591.9 (M+H)⁺.

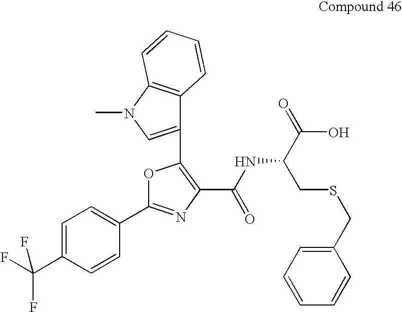

### (R)-3-Benzylsulfanyl-2-{[5-(1-methyl-1H-indol-3-yl)-2-(4-trifluoromethylphenyl)oxazole-4-carbonyl]amino}propionic acid

The title compound was prepared by a procedure analogous to that of Compound 1 by substituting, 1-methyl-1H-indole-3-carbonyl chloride for the 4-cyanobenzoyl chloride of Step A of Compound 1. MS 578.1 (M−H)⁻.

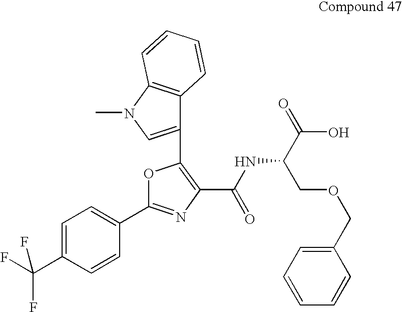

### (S)-3-Benzyloxy-2-{[5-(1-methyl-1H-indol-3-yl)-2-(4-trifluoromethylphenyl)oxazole-4-carbonyl]amino}propionic acid

The title compound was prepared by a procedure analogous to that of Compound 1 by substituting 1-methyl-1H-indole-3-carbonyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 564.0 (M+H)⁺.

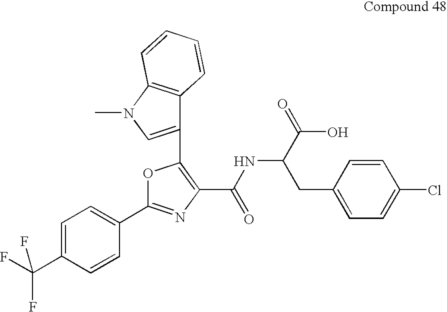

### 3-(4-Chlorophenyl)-2-{[5-(1-methyl-1H-indol-3-yl)-2-(4-trifluoromethylphenyl)oxazole-4-carbonyl]amino}propionic acid

The title compound was prepared by a procedure analogous to that of Compound 1 by substituting 1-methyl-1H-indole-3-carbonyl chloride for the 4-cyanobenzoyl chloride of Step A of Compound 1; and by substituting D,L-4-chlorophenylalanine ethyl ester hydrochloride for the H-Cys(Bzl)-L-OMe hydrochloride of Step D of Compound 1. MS 1134.0 (2M+H)⁺.

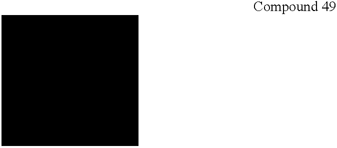

### (R)-3-Benzylsulfanyl-2-{[5-[4-(N-hydroxycarbamimidoyl)phenyl]-2-(4-trifluoromethylphenyl)oxazole-4-carbonyl]amino}propionic acid

### Step A: (R)-3-Benzylsulfanyl-2-{[5-[4-(N-hydroxycarbamimidoyl)phenyl]-2-(4-trifluoro methylphenyl)oxazole-4-carbonyl]amino}propionic acid methyl ester

A mixture of (R)-3-benzylsulfanyl-2-{[5-(4-cyanophenyl)-2-(4-trifluoro-methylphenyl)oxazole-4-carbonyl]amino}propionic acid methyl ester from Step D of Compound 1 (573 mg, 1 mmol), hydroxylamine hydrochloride (70 mg, 1 mmol) and triethylamine (0.21 mL, 1.5 mmol) in ethanol (5.0 mL) was heated at reflux temperature for 2 h. The reaction mixture was cooled to room temperature and passed through a sintered glass filter funnel. The off-white precipitate was washed with water and dried under vacuum giving 530 mg of title compound.

### Step B: (R)-3-Benzylsulfanyl-2-{[5-[4-(N-hydroxycarbamimidoyl)phenyl]-2-(4-trifluoromethylphenyl)oxazole-4-carbonyl]amino}propionic acid

To a solution of (R)-3-Benzylsulfanyl-2-{[5-[4-(N-hydroxycarbamimidoyl)-phenyl]-2-(4-trifluoromethylphenyl)oxazole-4-carbonyl]amino}propionic acid methyl ester from Step A (62 mg, 0.10 mmol) in THF (1.0 mL) was added 1N lithium hydroxide (aq., 0.5 mL) and the reaction mixture was stirred at room temperature for 3 h. After acidification with 1N HCl to pH 4-5, the white solid was collected by filtration giving 36 mg (62%) of the title compound. MS 585.0 (M+H)⁺.

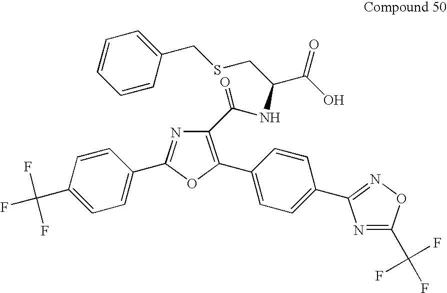

### (R)-3-Benzylsulfanyl-2-{[5-[4-(5-trifluoromethyl[1,2,4]oxadiazol-3-yl)phenyl]-2-(4-trifluoromethylphenyl)oxazole-4-carbonyl]amino}propionic acid

### Step A: (R)-3-Benzylsulfanyl-2-{[5-[4-(5-trifluoromethyl[1,2,4]oxadiazol-3-yl)phenyl]-2-(4-trifluoromethylphenyl)oxazole-4-carbonyl]amino}propionic acid methyl ester

A mixture of (R)-3-benzylsulfanyl-2-{[5-[4-(N-hydroxycarbamimidoyl)-phenyl]-2-(4-trifluoromethylphenyl)oxazole-4-carbonyl]amino}propionic acid methyl ester from Step A of Compound 49 (100 mg, 0.17 mmol) in trifluoroacetic anhydride (2.0 mL) was heated at reflux temperature for 30 min. The reaction mixture was cooled to room temperature and concentrated in vacuo. Purification by medium pressure liquid chromatography on silica gel (1:4 ethyl acetate/hexanes) gave 9 mg (8%) of the title compound. MS 677.1 (M+H)⁺.

### Step B: (R)-3-Benzylsulfanyl-2-{[5-[4-(5-trifluoromethyl[1,2,4]oxadiazol-3-yl)-phenyl]-2-(4-trifluoromethylphenyl)oxazole-4-carbonyl]amino}propionic acid

To a solution of (R)-3-benzylsulfanyl-2-{[5-[4-(5-trifluoromethyl[1,2,4]oxadiazol-3-yl)phenyl]-2-(4-trifluoromethylphenyl)oxazole-4-carbonyl]amino}propionic acid methyl ester from Step A (9 mg, 0.01 mmol) in THF (0.5 mL) was added 1N lithium hydroxide (aq., 0.3 mL) and the reaction mixture was stirred at room temperature for 3 h. After acidification with 1N HCl to pH 4-5, the white solid was collected by filtration giving 6 mg (90%) of the title compound. MS 664.0 (M+H)⁺.

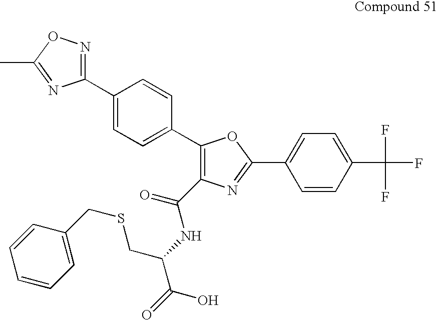

### (R)-3-Benzylsulfanyl-2-{[5-[4-(5-methyl[1,2,4]oxadiazol-3-yl)phenyl]-2-(4-trifluoromethylphenyl)oxazole-4-carbonyl]amino}propionic acid

The title compound was prepared by a procedure analogous to that of Compound 50 by substituting acetic anhydride for the trifluoroacetic anhydride of Step A of Compound 51. MS 609.0 (M+H)⁺.

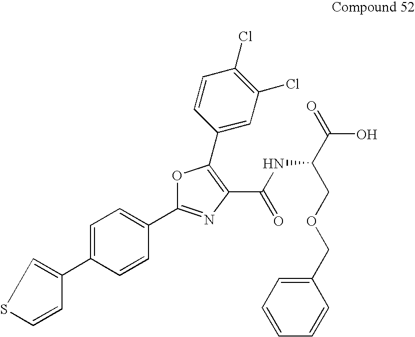

### (S)-3-Benzyloxy-2-{[5-(3,4-dichlorophenyl)-2-(4-thien-3-yl-phenyl)oxazole-4-carbonyl]amino}propionic acid

### Step A: 2-(4-Bromophenyl)-5-(3,4-dichlorophenyl)oxazole-4-carboxylic acid methyl ester

The title compound was prepared by a procedure analogous to that of Compound 1 by substituting 3,4-dichlorobenzoyl chloride for the 4-cyanobenzoyl chloride of Step A of Compound 1; and by substituting 4-bromo-1-iodobenzene for the 1-iodo-4-trifluoromethylbenzene of Step B of Compound 1.

### Step B: 5-(3,4-Dichlorophenyl)-2-(4-thien-3-yl-phenyl)oxazole-4-carboxylic acid methyl ester

A mixture of 2-(4-bromophenyl)-5-(3,4-dichlorophenyl)oxazole-4-carboxylic acid methyl ester from Step A (28 mg, 0.066 mmol), 3-thienylboronic acid (16.7 mg, 0.13 mmol), Pd(P(t-Bu)₃)₂ (0.013 mmol), Pd₂(dba)₃CHCl₃ (0.007 mmol) and potassium fluoride (11 mg, 0.19 mmol) in THF (1 mL) was heated at 60° C. for 10 h. The mixture was cooled to room temperature, filtered through a pad of silica gel and washed with EtOAc. The combined organic solution was concentrated in vacuo. Purification by medium pressure liquid chromatography on silica gel gave the title compound (10.5 mg, 0.0244 mmol, 37% yield). MS 882.7 (2M+Na)⁺.

### Step C: (S)-3-Benzyloxy-2-{[5-(3,4-dichlorophenyl)-2-(4-thien-3-yl-phenyl)oxazole-4-carbonyl]amino}propionic acid

The title compound was prepared by a procedure analogous to that of Steps C-E of Compound 1 by substituting 5-(3,4-dichlorophenyl)-2-(4-thien-3-yl-phenyl)oxazole-4-carboxylic acid methyl ester of Step B of Compound 52 for the 5-(4-cyanophenyl)-2-(4-trifluoromethylphenyl)oxazole-4-carboxylic acid methyl ester of Step C of Compound 1; and by substituting H-Ser(Bzl)-L-OMe hydrochloride for the H-Cys(Bzl)-L-OMe hydrochloride of Step D of Compound 1. MS 593.0 (M+H)⁺.

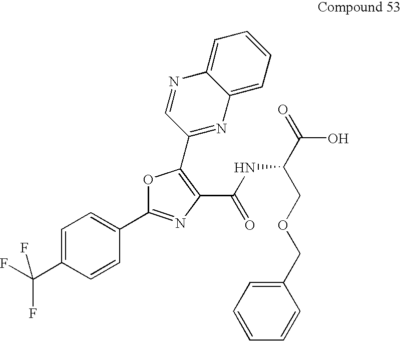

### (S)-3-Benzyloxy-2-{[5-quinoxalin-2-yl-2-(4-trifluoromethylphenyl)oxazole-4-carbonyl]amino}propionic acid

The title compound was prepared by a procedure analogous to that of Compound 1 by substituting quinoxaline-2-carbonyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 563.1 (M+H)⁺.

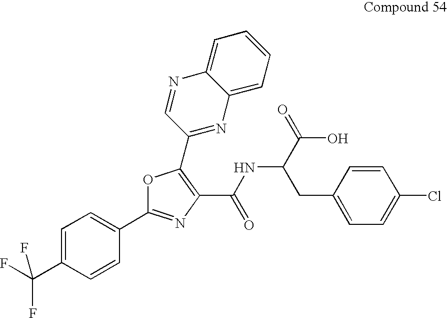

### 3-(4-Chlorophenyl)-2-{[5-quinoxalin-2-yl-2-(4-trifluoromethylphenyl)oxazole-4-carbonyl]amino}propionic acid

The title compound was prepared by a procedure analogous to that of Compound 1 by substituting quinoxaline-2-carbonyl chloride for the 4-cyanobenzoyl chloride of Step A of Compound 1; and by substituting D,L-4-chlorophenylalanine ethyl ester hydrochloride for the H-Cys(Bzl)-L-OMe hydrochloride of Step D of Compound 1. ¹H NMR (300 Hz, acetone-d₆ and CD₃OD) δ 3.32 (2H, d, J=3.4 Hz), 5.25 (H, t, J=4.5 Hz), 6.07 (2H, d, J=8.3 Hz), 7.16 (2H, d, J=8.3 Hz), 7.81 (2H, d, J=3.6 Hz), 7.93-7.99 (3H, m), 8.16 (1H, d, J=8.3 Hz), 8.54 (2H, d, J=8.3 Hz), 9,81 (1H, s).

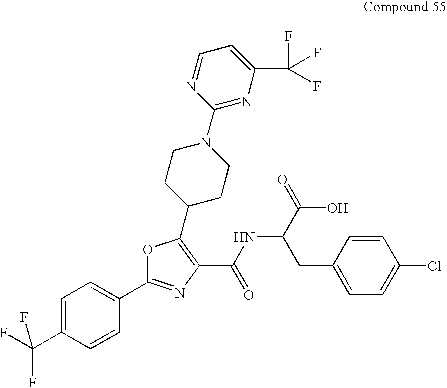

### 3-(4-Chlorophenyl)-2-({2-(4-trifluoromethylphenyl)-5-[1-(4-trifluoromethylpyrimidin-2-yl)piperidin-4-yl]oxazole-4-carbonyl}amino)propionic acid

The title compound was prepared by a procedure analogous to that of Compound 1 by substituting 1-(4-trifluoromethylpyrimidine-2-yl)piperidine-4-carbonyl chloride for the 4-cyanobenzoyl chloride of Step A of Compound 1; and by substituting D,L-4-chlorophenylalanine ethyl ester hydrochloride for the H-Cys(Bzl)-L-OMe hydrochloride of Step D of Compound 1. MS 669.0 (M+H)⁺.

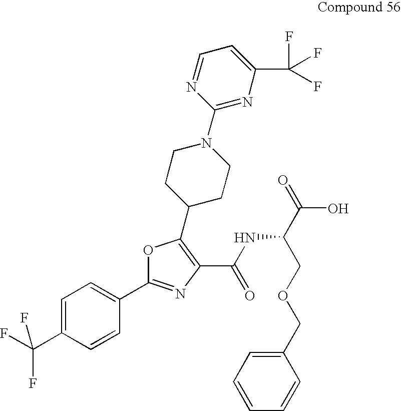

### (S)-3-Benzyloxy-2-({2-(4-trifluoromethylphenyl)-5-[1-(4-trifluoromethylpyrimidin-2-yl)piperidin-4-yloxazole-4-carbonyl}amino)propionic acid

The title compound was prepared by a procedure analogous to that of Compound 1 by substituting 1-(4-trifluoromethylpyrimidine-2-yl)piperidine-4-carbonyl chloride for the 4-cyanobenzoyl chloride of Step A of Compound 1; and by substituting H-Ser(Bzl)-L-OMe hydrochloride for the H-Cys(Bzl)-L-OMe hydrochloride of Step D of Compound 1. MS 664.0 (M+H)⁺.

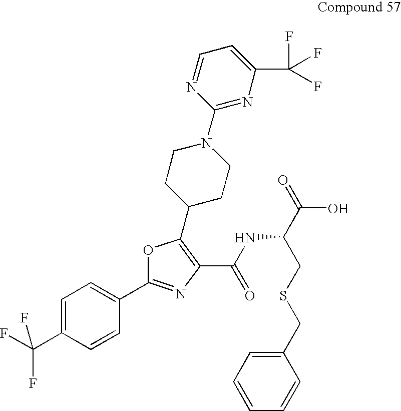

### (R)-3-Benzylsulfanyl-2-({2-(4-trifluoromethylphenyl)-5-[1-(4-trifluoromethylpyrimidin-2-yl)piperidin-4-yl]oxazole-4-carbonyl}amino)propionic acid

The title compound was prepared by a procedure analogous to that of Compound 1 by substituting 1-(4-trifluoromethylpyrimidine-2-yl)piperidine-4-carbonyl chloride for the 4-cyanobenzoyl chloride of Step A of Compound 1. MS 681.0 (M+H)⁺.

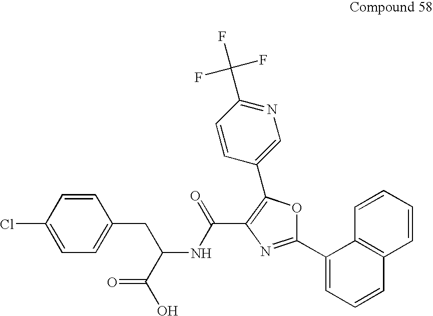

### 3-(4-Chlorophenyl)-2-{[2-naphthalen-1-yl-5-(6-trifluoromethylpyridin-3-yl)oxazole-4-carbonyl]amino}propionic acid

The title compound was prepared by a procedure analogous to that of Compound 1 by substituting 6-trifluoromethylnicotinoyl chloride for the 4-cyanobenzoyl chloride of Step A of Compound 1; and by substituting 1-iodonaphthalene for the 1-iodo-4-trifluoromethylbenzene of Step B of Compound 1; and by substituting D,L-4chlorophenylalanine ethyl ester hydrochloride for the H-Cys(Bzl)-L-OMe hydrochloride of Step D of Compound 1. MS 564.1 (M−H)⁻.

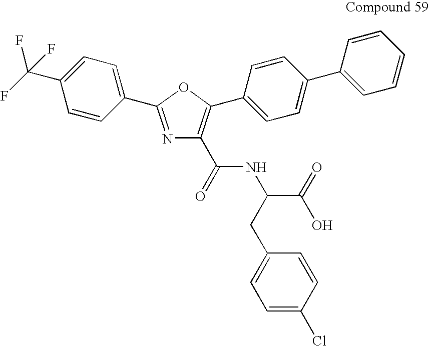

### 2-{[5-Biphenyl-4-yl-2-(4-trifluoromethylphenyl)oxazole-4-carbonyl]amino}-3-(4-chlorophenyl)propionic acid

The title compound was prepared by a procedure analogous to that of Compound 1 by substituting 4-biphenylcarbonyl chloride for the 4-cyanobenzoyl chloride of Step A of Compound 1; and by substituting D,L-4-chlorophenylalanine ethyl ester hydrochloride for the H-Cys(Bzl)-L-OMe hydrochloride of Step D of Compound 1. MS 592.0 (M+H)⁺.

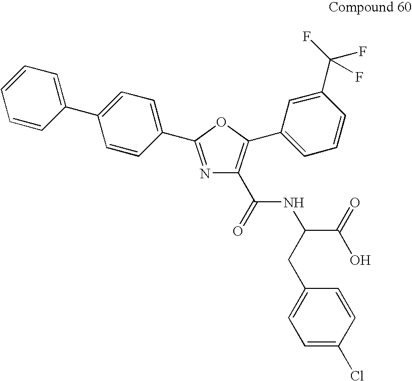

### 2-{[2-Biphenyl-4-yl-5-(3-trifluoromethylphenyl)oxazole-4-carbonyl]amino}-3-(4-chlorophenyl)propionic acid

The title compound was prepared by a procedure analogous to that of Compound 1 by substituting 3-trifluoromethylbenzoyl chloride for the 4-cyanobenzoyl chloride of Step A of Compound 1; and by substituting 4-bromobiphenyl for the 1-iodo-4-trifluoromethylbenzene of Step B of Compound 1; and by substituting D,L-4-chlorophenylalanine ethyl ester hydrochloride for the H-Cys(Bzl)-L-OMe hydrochloride of Step D of Compound 1. MS 592.0 (M+H)⁺.

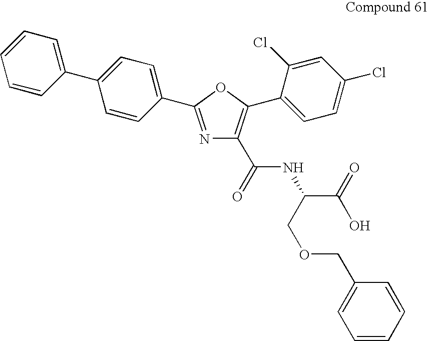

### (S)-3-Benzyloxy-2-{[2-biphenyl-4-yl-5-(2,4-dichlorophenyl)oxazole-4-carbonyl]amino}propionic acid

The title compound was prepared by a procedure analogous to that of Compound 1 by substituting 2,4-dichlorobenzoyl chloride for the 4-cyanobenzoyl chloride of Step A of Compound 1; and by substituting 4-bromobiphenyl for the 1-iodo-4-trifluoromethylbenzene of Step B of Compound 1; and by substituting H-Ser(Bzl)-L-OMe hydrochloride for the H-Cys(Bzl)-L-OMe hydrochloride of Step D of Compound 1. MS 589.0 (M+H)⁺.

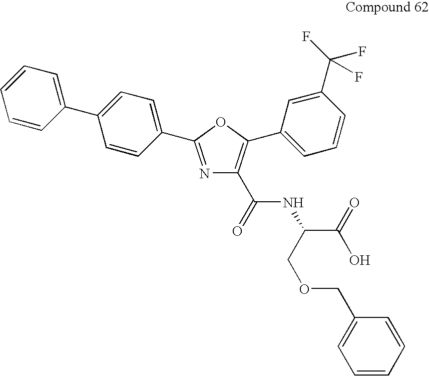

### (S)-3-Benzyloxy-2-{[2-biphenyl-4-yl-5-(3-trifluoromethylphenyl)oxazole-4-carbonyl]amino}propionic acid

The title compound was prepared by a procedure analogous to that of Compound 1 by substituting 3-trifluoromethylbenzoyl chloride for the 4-cyanobenzoyl chloride of Step A of Compound 1; and by substituting 4-bromobiphenyl for the 1-iodo-4-trifluoromethylbenzene of Step B of Compound 1; and by substituting H-Ser(Bzl)-L-OMe hydrochloride for the H-Cys(Bzl)-L-OMe hydrochloride of Step D of Compound 1. MS 587.1 (M+H)⁺.

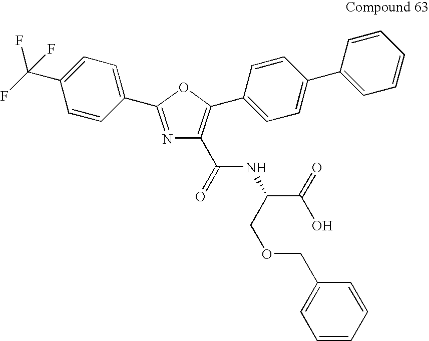

### (S)-3-Benzyloxy-2-{[5-biphenyl-4-yl-2-(4-trifluoromethylphenyl)oxazole-4-carbonyl]amino}propionic acid

The title compound was prepared by a procedure analogous to that of Compound 1 by substituting 4-biphenylcarbonyl chloride for the 4-cyanobenzoyl chloride of Step A of Compound 1; and by substituting H-Ser(Bzl)-L-OMe hydro-chloride for the H-Cys(Bzl)-L-OMe hydrochloride of Step D of Compound 1. MS 587.1 (M+H)⁺.

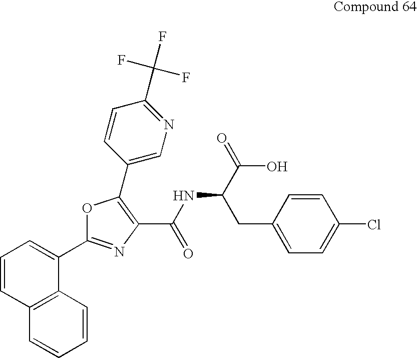

### (R)-3-(4-Chlorophenyl)-2-{[2-naphthalen-1-yl-5-(6-trifluoromethylpyridin-3-yl)oxazole-4-carbonyl]amino}propionic acid

The title compound was prepared by a procedure analogous to that of Compound 1 by substituting 6-trifluoromethylnicotinoyl chloride for the 4-cyanobenzoyl chloride of Step A of Compound 1; and by substituting 1-iodonaphthalene for the 1-iodo-4-trifluoromethylbenzene of Step B of Compound 1; and by substituting D-4-chlorophenylalanine methyl ester hydrochloride for the H-Cys(Bzl)-L-OMe hydrochloride of Step D of Compound 1. MS 564.1 (M−H)⁻.

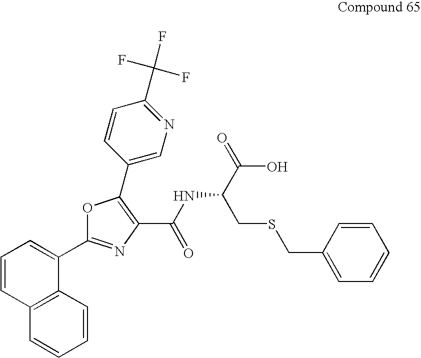

### (R)-3-Benzylsulfanyl-2-{[2-naphthalen-1-yl-5-(6-trifluoromethylpyridin-3-yl)oxazole-4-carbonyl]amino}propionic acid

The title compound was prepared by a procedure analogous to that of Compound 1 by substituting 6-trifluoromethylnicotinoyl chloride for the 4-cyanobenzoyl chloride of Step A of Compound 1; and by substituting 1-iodonaphthalene for the 1-iodo-4-trifluoromethylbenzene of Step B of Compound 1. MS 576.2 (M−H)⁻.

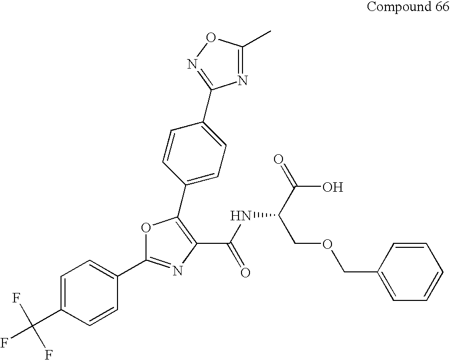

### (S)-3-Benzyloxy-2-{[5-[4-(5-methyl[1,2,4]oxadiazol-3-yl)phenyl]-2-(4-trifluoromethylphenyl)oxazole-4-carbonyl]amino}propionic acid

### Step A: 5-[4-(N-Hydroxycarbamimidoyl)phenyl]-2-(4-trifluoromethylphenyl)oxazole-4 carboxylic acid methyl ester

A mixture of 5-(4-Cyanophenyl)-2-(4-trifluoromethylphenyl)oxazole-4-carboxylic acid methyl ester from Step B of Compound 1 (532 mg, 1.43 mmol), hydroxylamine hydrochloride (99 mg, 1.43 mmol) and triethylamine (0.30 mL, 2.1 mmol) in ethanol (8.0 mL) was heated at reflux temperature for 3 h. The reaction mixture was cooled to room temperature and passed through a sintered glass filter funnel. The off-white precipitate was washed with water and dried under vacuum giving 401 mg (69%) of title compound. MS 406.0 (M+H)⁺.

### Step B: 5-[4-(N-Acetoxycarbamimidoyl)phenyl]-2-(4-trifluoromethylphenyl)oxazole-4 carboxylic acid methyl ester

To a mixture of 5-[4-(N-hydroxycarbamimidoyl)phenyl]-2-(4-trifluoromethyl-phenyl)oxazole-4-carboxylic acid methyl ester from Step A (401 mg, 1.0 mmol) in dichloromethane (8.0 mL) at 0° C. was added diisopropylethylamine (0.38 mL, 2.0 mmol) followed by acetyl chloride (0.092 mL, 1.3 mL), and the resulting reaction mixture was stirred at 0° C. for 1 h. and room temperature for 15 h. It was then concentrated in vacuo to give the crude ester. Purification by medium pressure liquid chromatography on silica gel (3:2 ethyl acetate/hexanes) gave 109 mg (24%) of the title compound. MS 448.0 (M+H)⁺.

### Step C: 5-[4-(5-Methyl[1,2,4]oxadiazol-3-yl)phenyl]-2-(4-trifluoromethylphenyl oxazole-4-carboxylic acid methyl ester

To solution of the product from Step B (100 mg, 0.22 mmol) in THF (3.0 mL) at room temperature was added TBAF (0.22 mL of 1M solution in THF), and the resulting reaction mixture was stirred at room temperature for 20 h. It was then concentrated in vacuo to give the crude product. Purification by medium pressure liquid chromatography on silica gel (1:5 ethyl acetate/hexanes) gave 50 mg (52%) of the title compound. MS 430.0 (M+H)⁺.

### Step D: (S)-3-Benzyloxy-2-{[5-[4-(5-methyl][1,2,4]oxadiazol-3-yl)]phenyl]-2-(4-trifluoro methylphenyl)oxazole-4-carbonyl]amino}propionic acid

The title compound was prepared by a procedure analogous to that of Steps C-E of Compound 1 by substituting 5-[4-(5-methyl[1,2,4]oxadiazol-3-yl)phenyl]-2-(4-trifluoromethylphenyl)oxazole-4-carboxylic acid methyl ester from Step C of Compound 66 for the 5-(4-cyanophenyl)-2-(4-trifluoromethylphenyl)-oxazole-4-carboxylic acid methyl ester of Step C of Compound 1; and by substituting H-Ser(Bzl)-L-OMe methyl ester hydrochloride for the H-Cys(Bzl)-L-OMe hydrochloride of Step D of Compound 1. MS 591.2 (M+H)⁺.

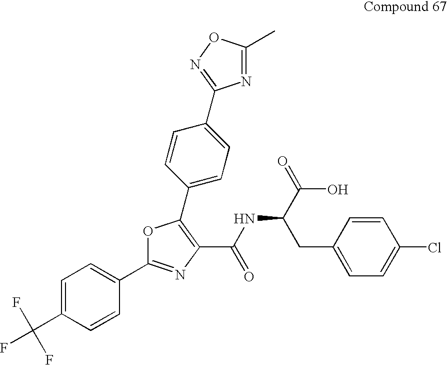

### 3-(4-Chlorophenyl)-2-{[5-[4-(5-methyl[1,2,4]oxadiazol-3-yl)phenyl]-2-(4-trifluoromethylphenyl)oxazole-4-carbonyl]amino}propionic acid

The title compound was prepared by a procedure analogous to that of Compound 66 by substituting D-4-chlorophenylalanine methyl ester hydrochloride for the H-Ser(Bzl)-L-OMe hydrochloride of Step D of Compound 66. MS 595.0 (M−H)⁻.

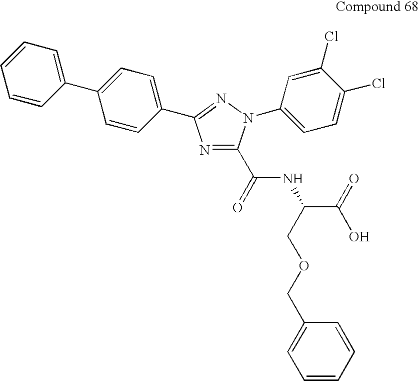

### (S)-3-Benzyloxy-2-{[5-biphenyl-4-yl-2-(3,4-dichlorophenyl)-2H-[1,2,4]triazole-3-carbonyl]amino}propionic acid

The title compound was prepared by a procedure analogous to that of Compound 4 by substituting 3,4-dichlorophenylhydrazine for the 3-trifluoromethylphenylhydrazine of Step C of Compound 4; and by substituting H-Ser(Bzl)-L-OMe hydrochloride for the D-4-chlorophenylalanine methyl ester hydrochloride of Step F of Compound 4. MS 587.0 (M+H)⁺.

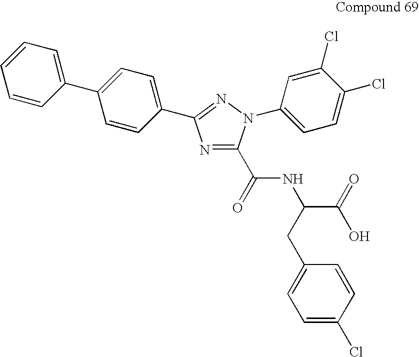

### 2-{[5-Biphenyl-4-yl-2-(3,4-dichlorophenyl)-2H-[1,2,4]triazole-3-carbonyl]amino}-3-(4-chlorophenyl)propionic acid

The title compound was prepared by a procedure analogous to that of Compound 4 by substituting 3,4-dichlorophenylhydrazine for the 3-trifluoromethylphenylhydrazine of Step C of Compound 4; and by substituting D,L-4-chlorophenylalanine ethyl ester hydrochloride for the D-4-chlorophenylalanine methyl ester hydrochloride of Step F of Compound 4. MS 589.0 (M−H)⁻.

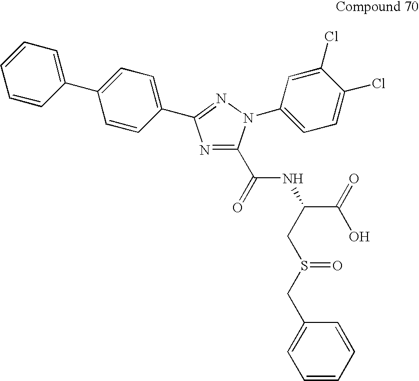

### (R)-2-{[5-Biphenyl-4-yl-2-(3,4-dichlorophenyl)-2H-[1,2,4]triazole-3-carbonyl]amino}-3-phenylmethanesulfinylpropionic acid

The title compound was prepared by a procedure analogous to that of Compound 4 by substituting 3,4-dichlorophenylhydrazine for the 3-trifluoromethylphenylhydrazine of Step C of Compound 4; and by substituting H-Cys(Bzl)-L-OMe hydrochloride for the D-4-chlorophenylalanine methyl ester hydrochloride of Step F of Compound 4. MS 617.0 (M−H)⁻.

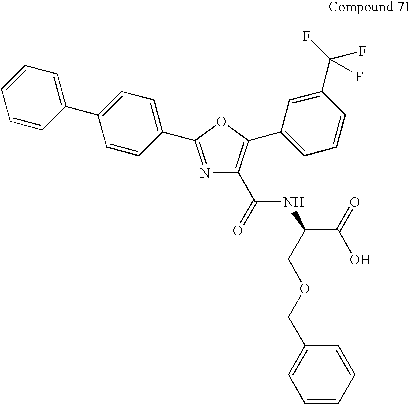

### (R)-3-Benzyloxy-2-{[2-biphenyl-4-yl-5-(3-trifluoromethylphenyl)oxazole-4-carbonyl]amino}propionic acid

The title compound was prepared by a procedure analogous to that of Compound 1 by substituting 3-trifluoromethylbenzoyl chloride for the 4-cyanobenzoyl chloride of Step A of Compound 1; by substituting 4-bromobiphenyl for the 1-iodo-4-trifluoromethylbenzene of Step B of Compound 1, and by substituting H-Ser(Bzl)-D-OMe hydrochloride for the H-Cys(Bzl)-L-OMe hydrochloride of Step D of Compound 1. MS 587.1 (M+H)⁺.

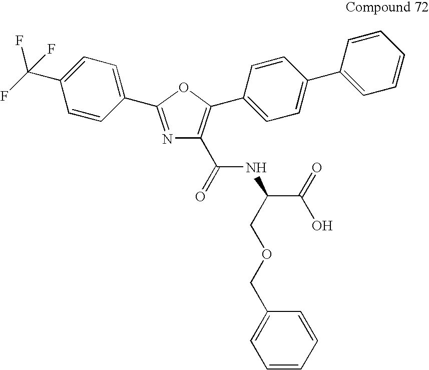

### (R)-3-Benzyloxy-2-{[5-biphenyl-4-yl-2-(4-trifluoromethylphenyl)oxazole-4-carbonyl]amino}propionic acid

The title compound was prepared by a procedure analogous to that of Compound 1 by substituting biphenyl-4-carbonyl chloride for the 4-cyanobenzoyl chloride of Step A of Compound 1; and by substituting H-Ser(Bzl)-D-OMe hydrochloride for the H-Cys(Bzl)-L-OMe hydrochloride of Step D of Compound 1. MS 587.1 (M+H)⁺.

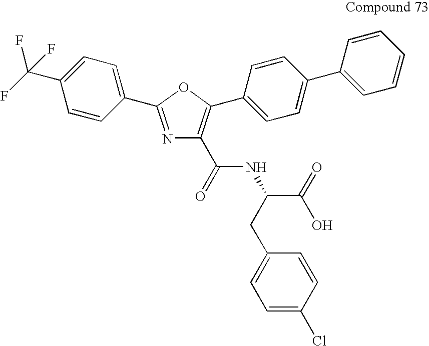

### (S)-2-{[5-Biphenyl-4-yl-2-(4-trifluoromethylphenyl)oxazole-4-carbonyl]amino}-3-(4-chlorophenyl)propionic acid

The title compound was prepared by a procedure analogous to that of Compound 1 by substituting biphenyl-4-carbonyl chloride for the 4-cyanobenzoyl chloride of Step A of Compound 1; and by substituting L-4-chlorophenylalanine methyl ester hydrochloride for the H-Cys(Bzl)-L-OMe hydrochloride of Step D of Compound 1. MS 592.0 (M+H)⁺.

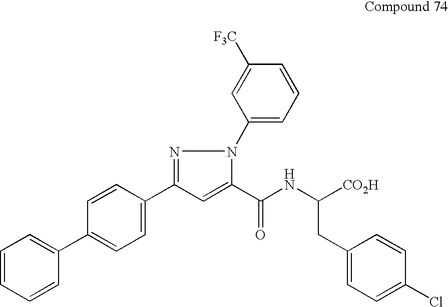

### 2-{[5-Biphenyl-4-yl-2-(3-trifluoromethylphenyl)-2H-pyrazole-3-carbonyl]amino}-3-(4-chlorophenyl)propionic acid

### Step A: 4-Biphenyl-4-yl-2,4-dioxobutyric acid ethyl ester

Lithium bis(trimethylsilyl)amide (1 N in THF, 30.6 ml, 30.6 mmol) was cooled to −78° C. and a dichlqromethane solution of 1-biphenyl-4-yl-ethanone was added under N₂. After 45 min., 6 g of diethyl oxalate was added. The reaction was let warm to room temperature slowly, and was stirred for an additional 16 h while a light yellow precipitate formed. To the reaction mixture was added 20 ml of saturated NH₄Cl solution, and the precipitate was filtered and dried to give the title compound (9.94 g, 90% purity, quantitative yield). MS 319.1 (M+Na)⁺.

### Step B: 5-Biphenyl-4-yl-2-(3-trifluoromethylphenyl)-2H-pyrazole-3-carboxylic acid ethyl ester

To a solution of 4-Biphenyl-4-yl-2,4-dioxobutyric acid ethyl ester from Step A (1.0 g, 3.376 mmol) in acetic acid (10 mL) was added 3-trifluoromethylphenylhydrazine (0.60 g, 3.376 mmol) and the reaction mixture was stirred at room temperature for 15 h. To the reaction mixture was added water, at which point a white precipitate formed and was filtered and washed with water. It was then dissolved in ethyl acetate and hexane and concentrated in vacuo. Purification of the crude product by medium pressure liquid chromatography on silica gel gave the title compound as a major product. Regio-isomer 5-biphenyl-4-yl-1-(3-trifluoromethyl-phenyl)-1H-pyrazole-3-carboxylic acid ethyl ester was isolated as a minor product from this reaction.

### Step C: 5-Biphenyl-4-yl-2-(3-trifluoromethylphenyl)-2H-pyrazole-3-carboxylic acid.

5-Biphenyl-4-yl-2-(3-trifluoromethylphenyl)-2H-pyrazole-3-carboxylic acid ethyl ester from Step B was dissolved in THF (10 mL), followed by addition of 1N NaOH (10 ml). The reaction mixture was stirred at room temperature for 10 h. After THF was removed, the aqueous solution was acidified with 1N HCl to pH 3. A white precipitate formed and was filtered and washed with water and then hexane. The solid was dried under vacuum to give 5-biphenyl-4-yl-2-(3-trifluoromethylphenyl)-2H-pyrazole-3-carboxylic acid (1.23 g, 3.00 mmol, 89% yield over two steps). MS 431.1 (M+Na)⁺.

### Step D: 2-{[5-Biphenyl-4-yl-2-(3-trifluoromethylphenyl)-2H-pyrazole-3-carbonyl]-amino}-3-(4-chlorophenyl)propionic acid ethyl ester

To a mixture of 5-Biphenyl-4-yl-2-(3-trifluoromethylphenyl)-2H-pyrazole-3-carboxylic acid from Step C (0.345 g, 0.846 mmol), D,L-4-chlorophenylalanine ethyl ester hydrochloride (0.223 g, 0.846 mmol), and EDCI (0.162 g, 0.846 mmol) in anhydrous THF (5.0 mL) was added triethylamine (0.24 mL, 1.69 mmol) and the resulting reaction mixture was stirred at room temperature for 15 h. After solvent was removed, the crude product was taken up in 1N HCl. A white precipitate formed and was filtered. The solid was then washed with additional 1N HCl followed by water, ether and hexane. Purification by medium pressure liquid chromatography on silica gel gave the title compound. (0.183 g, 0.296 mmol, 35% yield). MS 618.2 (M+H)⁺.

### Step E: 2-{[5-Biphenyl-4-yl-2-(3-trifluoromethylphenyl)-2H-pyrazole-3-carbonyl]amino}-3-(4-chlorophenyl)propionic acid

2-{[5-Biphenyl-4-yl-2-(3-trifluoromethylphenyl)-2H-pyrazole-3-carbonyl]-amino}-3-(4-chlorophenyl)-propionic acid ethyl ester from Step D (20 mg, 0.032 mmol) was dissolved in THF (2 mL) followed by the addition of 1N NaOH (0.8 mL), and the reaction mixture was stirred for 10 h at room temperature. After THF was removed, the aqueous phase was acidified with 1N HCl to pH3, while a light yellow precipitate formed. The precipitate was then filtered, washed with water (5 mL) and ether/hexane (1:2, 10 ml) to give the title compound as a white solid (4.7 mg, 0.008 mmol, 25% yield). MS 590.0 (M+H)⁺.

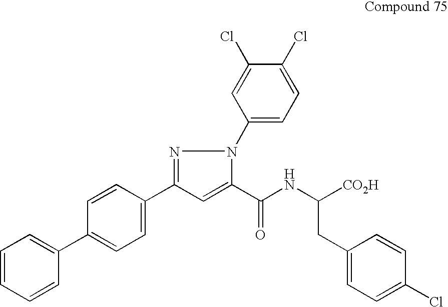

### 2-{[5-Biphenyl-4-yl-2-(3,4-dichlorophenyl)-2H-pyrazole-3-carbonyl]amino}-3-(4-chlorophenyl)propionic acid

The title compound was prepared by a procedure analogous to that of Compound 74 by substituting 3,4-dichlorophenylhydrazine for the 3-trifluoromethylphenylhydrazine of Step B of Compound 74. MS 591.0 (M+H)⁺.

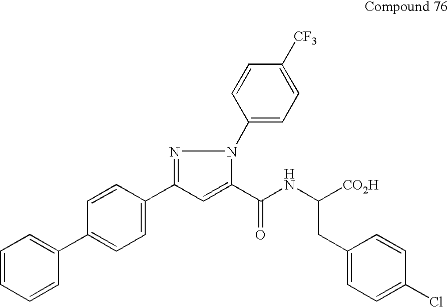

### 2-{[5-Biphenyl-4-yl-2-(4-trifluoromethylphenyl)-2H-pyrazole-3-carbonyl]amino}-3-(4-chlorophenyl)propionic acid

The title compound was prepared by a procedure analogous to that of Compound 74 by substituting 4-trifluoromethylphenylhydrazine for the 3-trifluoromethylphenylhydrazine of Step B of Compound 74. MS 590.0 (M+H)⁺.

### Example 2

### Assay to Evaluate Effect on Type III Protein Secretion Systems

The ability of the compounds of the invention to inhibit Type III protein secretion systems may be analyzed as follows.

Primary assay: Type III protein secretion of the chimeric SopE′-'Bla polypeptide by Salmonella enterica. This procedure is a cell-based assay that measures the type III-dependent secretion by Salmonella enterica of a plasmid-encoded chimeric polypeptide whose synthesis can be regulated, and which is endowed with an enzymatic activity that can be monitored calorimetrically by hydrolysis of a substrate that is unable to penetrate into the bacterial cytoplasm within the time constraints of the reaction. Thus, the colorimetric reaction is not influenced by SopE′-'Bla polypeptide in the bacterial cytoplasm. Instead, it effectively measures the amount of polypeptide that has been secreted from the S. enterica cytoplasm to the extracellular medium via type III system protein secretion.

The SopE′-' Bla recombinant polypeptide consists of two functionally distinct domains spliced together. The N-terminus domain is encoded by a polynucleotide region specifying the signal for type III secretion of the SopE polypeptide of S. enterica, an effector of the SPI1 type III protein secretion system. The C-terminus domain of SopE′-'Bla consists of a 263 amino acid peptide sequence that corresponds to the TEM-1 β-lactamase expressed by plasmid pBR322 but without its N-terminal signal sequence. The TEM-1 β-lactamase part of the SopE′-'Bla chimeric polypeptide is used as a reporter enzyme. It is capable of hydrolyzing nitrocefin resulting in a product whose accumulation can be monitored by colorimetric detection. The secretion of the SopE′-'Bla chimeric polypeptide from the cytoplasm to the extracellular medium is dependent on type III protein secretion.

For this procedure, cells grown under conditions known to favor a functional SPI1 secretion system are induced for expression of the SopE′-'Bla protein and grown either in the presence or in the absence of putative inhibitors for determined time. Nitrocefin is then added to the various cultures and its hydrolysis are used for quantitation. An inhibitor of Type III protein secretion is generally a compound that reduces the signal of the enzymatic reaction by decreasing the amount of SopE′-'Bla secreted into the extracellular medium.

Secondary assay: Type III-dependent protein secretion of the SipB polypeptide by S. enterica. The SipB protein of S. enterica is another effector of the SPI1 type III protein secretion system from S. enterica. In this cell-based procedure, the Type III-dependent secretion of SipB from the bacterial cytoplasm to the extracellular medium was measured through its reactivity with a cognate mouse monoclonal.

Salmonella enterica cells growing either in the presence or in the absence of inhibitors are induced for the production of SipB. Following an established period of growth the cells are sedimented and the amount of SipB present in the supernatant is quantified with a scanning imager following application of immunoblot techniques. Detection may employ an anti-SipB mouse monoclonal antibody (e.g., obtained from Jorge Galan, SUNY at Stony Brook, N.Y.) followed by treatment with commercially available sheep anti-mouse polyclonal antibody conjugated with horseradish peroxidase. Thereafter the membrane is treated with a peroxidase chemiluminescent substrate and exposed to film for an appropriate exposure time. Inhibition may be measured relative to untreated controls.

Tertiary assay: inhibition of Type III protein secretion of effectors from a Pseudomonas aeruginosa system. Type III protein secretion is used by P. aeruginosa to secrete several essential virulence determinants. One effector of the type III protein secretion system of P. aeruginosa PA103 is the virulence determinants ExoU.

The amount of Type III-dependent secretion of ExoU by P. aeruginosa PA103 can be determined in a cell-based assay by quantification of the 73.9 kDa ExoU protein scereted into the extracellular medium. Such quantitation can be achieved by growing strain PA103 in a deferrated medium in the presence of nitrilotriacetic acid (an inducer of Type III protein secretion in P. aeruginosa) and either in the presence or absence of putative inhibitors. After a prolonged growth period, the cells are sedimented and the supernatants concentrated by ammonium sulfate precipitation. The proteins in the resuspended pellets are separated by electrophoresis on SDS-polyacrylamide gels. After staining gels with Colloidal Blue™, the 73.9 kDa ExoU band is quantitated by scanning through an imager. The effects of inhibitors on the intensity of the ExoU band may be measured relative to that of untreated controls.

By way of example, assay results for preferred compounds of the invention are provided below in Table I.

TABLE I

| | ExoU | | ExoU | SipB | SopE |
|---|---|---|---|---|---|
| | IC50 | ExoU % | Conc | IC50 | IC50 |
| Compound | (μM) | Inh | (μM) | (μM) | (μM) |
| 1 | 70.7 | | | | |
| 2 | | | | | 16.5 |
| 3 | 20.6 | | | | |
| 4 | 56.6 | | | | |
| 5 | 12.5 | | | | |
| 6 | | | | | 25.6 |
| 7 | 54.4 | | | | |
| 8 | 45.4 | | | | |
| 9 | 26.7 | | | | |
| 10 | 42.4 | | | | |
| 11 | | | | | 42.2 |
| 12 | | 84.3 | 100 | | 14.0 |
| 13 | 19.3 | | | | |
| 14 | 62.8 | | | | |
| 15 | 79.9 | | | | |
| 16 | 80.9 | | | | |
| 17 | 34.3 | | | | |
| 18 | 89.1 | | | | |
| 19 | 78.2 | | | | |
| 20 | | | | | 3.3 |
| 21 | | | | | 3.1 |
| 22 | 82.4 | | | | |
| 23 | 40.7 | | | | |
| 24 | | | | | 80.5 |
| 25 | | | | | 62.6 |
| 26 | | | | | 5.8 |
| 27 | 93.1 | | | | |
| 28 | | | | | 3.6 |
| 29 | 73.4 | | | | |
| 30 | | | | | 29.2 |
| 31 | | | | | 71.5 |
| 32 | | | | | 77.3 |
| 33 | 48.9 | | | | |
| 34 | 47.4 | | | | |
| 35 | 79.8 | | | | |
| 36 | | | | | 3.3 |
| 37 | 91.7 | | | | |
| 38 | | | | | 2.4 |
| 39 | | | | | 9.1 |
| 40 | | | | | 5.2 |
| 41 | 51.6 | | | | |
| 42 | 27.1 | | | | |
| 43 | 45.7 | | | | |
| 44 | | | | | 3.3 |
| 45 | 51.8 | | | | |
| 46 | | | | | 47.3 |
| 47 | | | | | 54.0 |
| 48 | 73.5 | | | | |
| 49 | | | | | 34.7 |
| 50 | | | | | 64.3 |
| 51 | 19.5 | | | | |
| 52 | 56.4 | | | | |
| 53 | 64.6 | | | | |
| 54 | | | | | 49.4 |
| 55 | 51.7 | | | | |
| 56 | | | | | 12.0 |
| 57 | 50.0 | | | | |
| 58 | | | | | 4.2 |
| 59 | | 84.1 | 100 | | |
| 60 | | 90.2 | 100 | | |
| 61 | 66.1 | | | | |
| 62 | 50.5 | | | | |
| 63 | 37.3 | | | | |
| 64 | 51.4 | | | | |
| 65 | 53.1 | | | | |
| 66 | 12.5 | | | | |
| 67 | 12.5 | | | | |
| 68 | 39.2 | | | | |
| 69 | 32.4 | | | | |
| 70 | 25.5 | | | | |
| 71 | 45.3 | | | | |
| 72 | 54.8 | | | | |
| 73 | 56.1 | | | | |
| 74 | | | | | 1.5 |
| 75 | | | | | 3.0 |
| 76 | | | | | 19.6 |

All publications and patent applications cited herein are incorporated by reference to the same extent as if each individual publication or patent application was specifically and individually indicated to be incorporated by reference.

Allthough certain embodiments have been described in detail above, those having ordinary skill in the art will clearly understand that many modifications are possible in the embodiments without departing from the teachings thereof. All such modifications are intended to be encompassed within the claims of the invention.

### REFERENCES

1. Oxazole Derivatives, Their Preparation and Their Use as Cytokine Inhibitors, Naruto, S., Sugano, Y., Tatsuta, T., Burdi, D., Porte, A., Grisostomi, C., WO 02/064588.
2. Preparation of 1H-Indole-3-carboxamides, 1H-Indazole-3-carboxamide, 1H-Pyrido[4,3-b]indole-1-ones and Pyrrolo[1,2,3-de]-1,4-benzoxazine-6-carboxamides as Cannabinoid Receptor Modulators for Treating Respiratory and Non-respiratory Diseases, Leftheris, K., Zhao, R., Chen, B., Kiener, P., Wu, H., Pendit, C. R., Wrobleski, S., Chen, P., Hynes, J., Longphre, M., Norris, D., Spergel, S., Tokarski, J., WO 2001/058869.
3. New Convenient Synthesis of α-C-Acylamino Acids and α-Amino Ketones, Suzuki, M., Iwasaki, T., Miyoshi, M., Okumura, K., Matsumoto, K., J. Org. Chem, 1973, 38, 3571.
4. The Synthesis of 2-Azapodophyllotoxins, Bosmans, J. P., Eycken, J., Vandewalle, M., Tetrahedron Lett. 1989, 30, 3877.

1. A compound of formula I: 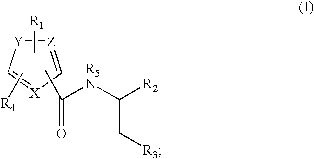
wherein X is N; Y is O, S or NRₐ; and Z is CHₙ, or N; n is 0 or 1; and X, Y and Z taken together with the carbon atoms to which they are attached form an oxazole, triazole, thiazole, or imidazole ring;
Rₐ is hydrogen or R₄;
R₁ is aryl, substituted aryl, aryl-(C₂-C₄alkynyl), heteroaryl, substituted heteroaryl, heterocyclyl, or substituted heterocyclyl;
R₂ is H or carboxy;
R₃ is aryl, optionally substituted by one or more halogen atoms; benzyloxy; benzylthio; benzylsulfinyl; benzylsulfonyl; and
R₄ is aryl or substituted aryl;
R₅ is hydrogen or lower alkyl;
when Rₐ is R₁ and Z is CHₙ, then n is 1, and when Ra is H, then Z is CHₙ and n is 0;
or an optical isomer, diastereomer or enantiomer thereof; or a pharmaceutically acceptable salt, hydrate, or prodrug thereof.

2. A method of treating a patient with an infection due to Gram-negative pathogenic bacteria by administering Type III protein secretion inhibitors having the formula (II): 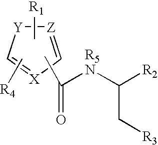
wherein X is N or CH; Y is O, S or NRₐ; and Z is CHₙ or N; n is 0 or 1; and X, Y and Z taken together with the carbon atoms to which they are attached form an oxazole, triazole, thiazole, or imidazole ring;
Rₐ is hydrogen or R₄;
R₁ is aryl, substituted aryl, aryl-(C₂-C₄alkynyl), heteroaryl, substituted heteroaryl, heterocyclyl, or substituted heterocyclyl;
R₂ is H or carboxy;
R₃ is aryl, optionally substituted by one or more halogen atoms; benzyloxy; benzylthio; benzylsulfinyl; benzylsulfonyl; and
R₄ is aryl or substituted aryl;
R₅ is hydrogen or lower alkyl;
when Rₐ is R₁ and Z is CHₙ, then n is 1, and when Ra is H, then Z is CHₙ and n is 0;
or an optical isomer, diastereomer or enantiomer thereof; or a pharmaceutically acceptable salt, hydrate, or prodrug thereof.

3. The compound of claim 1 wherein X is N, Y is 0, Z is C, and R₁ is bonded to Z.

4. The compound of claim 1 wherein R₁ is selected from the group consisting of two ring fused heterocyclyl.

5. The compound of claim 3 wherein R₁ is benzothienyl, benzofuryl, N-methylindolyl, quinolinyl, or quinoxalinyl.

6. The compound of claim 1 wherein R₁ is biphenyl, or mono- or disubstituted phenyl.

7. The compound of claim 1 wherein R₁ is substituted pyridinyl, or substituted piperidinyl.

8. The compound of claim 1 wherein R₃ is benzyloxy, benzylthio, chlorophenyl, benzylsulfonyl, or benzylsulfinyl.

9. The compound of claim 1 wherein R₄ is substituted phenyl or naphthyl.

10. The compound of claim 1 having the formula:

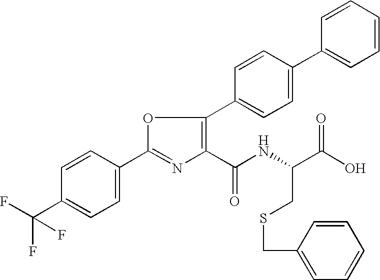

11. The compound of claim 1 having the formula:

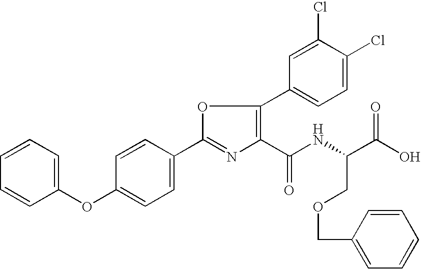

12. The compound of claim 1 having the formula:

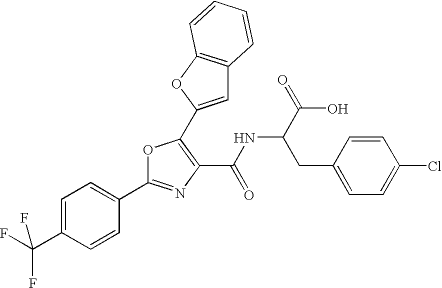

13. The compound of claim 1 having the formula:

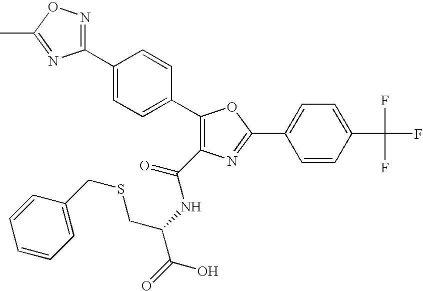

14. The compound of claim 1 having the formula:

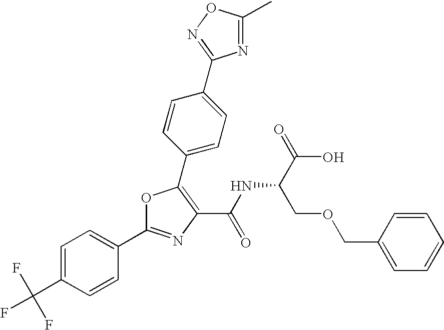

15. The compound of claim 1 having the formula:

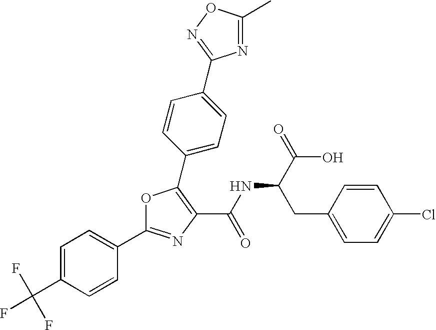

16. A method of inhibiting bacteria with Type III protein secretion systems, said method comprising administration of an effective amount of a compound according to claim 1 to a subject in need of treatment for infection by said bacteria with Type III protein secretion systems.
